(19) United States
(12) Patent Application Publication
(10) Pub. No.: US 2024/0389531 A1
(43) Pub. Date: Nov. 28, 2024

(54) PERONOSPORA RESISTANT SPINACH

(71) Applicant: Syngenta Crop Protection AG, Basel (CH)

(72) Inventors: Anoma Lokossou, Enkhuizen (NL); Michel de Lange, Enkhuizen (NL)

(73) Assignee: Syngenta Crop Protection AG, Basel (CH)

(21) Appl. No.: 18/692,609

(22) PCT Filed: Dec. 5, 2022
(86) PCT No.: PCT/EP2022/084413

§ 371 Date: Mar. 15, 2024

(30) Foreign Application Priority Data
Dec. 7, 2021 (EP) 21212976.1

(51) Int. Cl. A01H 6/02; A01H 1/00; A01H 1/04; A01H 5/12; C12Q 1/6895

(52) U.S. Cl. CPC A01H 6/028; A01H 1/04; A01H 1/1245; A01H 5/12; C12Q 1/6895; C12Q 2600/13; C12Q 2600/156

(57) ABSTRACT

The present invention relates to spinach plants displaying resistance to Peronospora effusa. The present invention also relates to seeds and parts of said plants, for example leaves and heads. The present invention further relates to methods of making and using such seeds and plants. The present invention also relates to genetic sequences associated with said resistance to Peronospora effusa and to molecular markers associated with said genetic sequences.

### RELATED APPLICATION INFORMATION

This application claims the benefit of European Patent Application No. 21212976.1, filed Dec. 7, 2021, the disclosure of which is incorporated by reference herein in its entirety.

### FIELD OF THE INVENTION

The present invention relates to novel spinach plants exhibiting resistance to Peronospora effusa. The present invention also relates to seeds and parts of said plants, for example leaves. The present invention further relates to methods of making and using such seeds and plants. The present invention also relates to novel genetic sequences associated with said resistance to Peronospora effusa and to molecular markers associated with said novel genetic sequences.

### BACKGROUND OF THE INVENTION

Plant pathogens are known to cause massive damage to important crops, resulting in significant agricultural losses with widespread consequences for both the food supply and other industries that rely on plant materials. As such, there is a long felt need to reduce the incidence and/or impact of agricultural pests on crop production.

An example of such a pathogen is the fungus Peronospora effusa, which causes downy mildew in spinach, one of the most economically important diseases in this crop. P. effusa occurs worldwide and represents a significant problem, impacting both the yield and quality of cultivated spinach (Spinacia oleracea).

Peronospora effusa is an obligate pathogen and belongs to the group Oomycetes, a class of relatively primitive fungi. Other members of this group are, for instance, Pythium and Phytophthora. High humidity conditions are favorable for P. effusa sporangia development, which disperse by wind and rain, with the potential for widespread infection. Sexual reproduction of the pathogen results in oospore formation, which can persist in the environment (e.g., soil) or in seeds.

Symptoms of downy mildew caused by P. effusa in infected spinach plants include yellow, irregular, chlorotic lesions on the leaves, which are not marketable. Management of P. effusa is primarily via the development and introduction of new resistant spinach varieties and agricultural practices such as crop rotation, clearing of plant debris, and reduction of moisture on the leaves.

There remains a need for novel resistances against the fungal pathogen Peronospora effusa, in particular, broad spectrum resistance against this fungal pathogen.

### SUMMARY OF THE INVENTION

The present invention addresses the need for additional and improved resistances to Peronospora effusa by providing novel spinach plants, and parts and seeds thereof, comprising Peronospora resistance traits designated as “ADQ” and “ADZ”. Also provided are nucleic acid markers for identifying and producing spinach plants (e.g., S. oleracea plants), and parts and seeds thereof, comprising one or both of the Peronospora resistance traits. The ADQ and ADZ Peronospora resistances of the invention combine Peronospora resistances from a wild Spinacia turkestanica source and a S. oleracea source and have been localized to a small region of the chromosome that maintains the full resistance spectra of both sources without significant linkage drag and without adverse phenotypes (e.g., dwarfism). The ADQ and ADZ Peronospora resistance-conferring introgressed sequences, located on chromosome 3, are of a dominant nature; hence one copy of the sequence provides a Peronospora resistance phenotype, which is qualitative in nature and segregates in a monogenic manner.

Altogether, the characteristics of the Peronospora resistant spinach plants of the present invention provide a spinach grower with novel solutions to enhance economic and commercial efficiency when deploying spinach varieties in a Peronospora pressured field.

In a first embodiment, the invention provides a spinach plant (e.g., a cultivated spinach plant, such as a Spinacia oleracea plant) resistant to Peronospora effusa comprising an introgressed sequence that confers a qualitative and dominant resistance to Peronospora effusa, wherein said introgressed sequence is located on chromosome 3, and wherein the resistance of the plant to Peronospora effusa is enhanced as compared with a control S. oleracea plant lacking said introgressed sequence. Optionally, said introgressed sequence comprises a G genotype (i.e., the resistance-associated genotype) in the heterozygous or homozygous state for SNP marker 11 in SEQ ID NO: 55.

In a further embodiment, the invention provides a spinach plant (e.g., a cultivated spinach plant, such as a Spinacia oleracea plant) with enhanced resistance to Peronospora effusa comprising an introgressed sequence that confers a qualitative and dominant resistance to Peronospora effusa, wherein said introgressed sequence is located on chromosome 3, and wherein the resistance of the plant to Peronospora effusa is enhanced as compared with a control S. oleracea plant lacking said introgressed sequence. Optionally, said introgressed sequence comprises a G genotype in the heterozygous or homozygous state for SNP marker 11 in SEQ ID NO: 55.

In embodiments, the introgressed sequence comprises the Peronospora resistance-associated genotype/allele at one or more of the following SNP markers, optionally in addition to the G genotype in the heterozygous or homozygous state for SNP marker 11 in SEQ ID NO: 55:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45; and/or
j) an A genotype in the heterozygous or homozygous state for SNP marker 10 in SEQ ID NO: 50.

In embodiments, the introgressed sequence comprises the Peronospora resistance-associated genotype at two or more, three or more, four or more, five or more, six or more, or seven or more of the SNP markers of c) to j) in any combination, optionally at all of the SNP markers of c) to j). In embodiments, the introgressed sequence comprises the Peronospora resistance-associated genotype at two or more, three or more, four or more, five or more, six or more, seven or more, eight or more, or nine or more of the SNP markers of a) to j) in any combination, optionally at all of the SNP markers of a) to j).

The introgressed sequence can comprise any combination of the resistance-associated genotypes at SNP markers 11, 10, 9, 8, 7, 6, 5, 4, 3, 2 and/or 1. In embodiments, the introgressed sequence comprises the resistance-associated genotypes at any of the following combinations of SNP markers:
i. 11 and 1;
ii. 11 and 2;
iii. 11 and 3;
iv. 11 and 4;
v. 11 and 5;
vi. 11 and 6;
vii. 11 and 7;
viii. 11 and 8;
ix. 11, 10, 9 and 8;
x. 11, 10, 9, 8 and 7;
xi. 11, 10, 9, 8, 7 and 6;
xii. 11, 10, 9, 8, 7, 6 and 5;
xiii. 11, 10, 9, 8, 7, 6, 5 and 4;
xiv. 11, 10, 9, 8, 7, 6, 5, 4 and 3;
xv. 11, 10, 9, 8, 7, 6, 5, 4, 3 and 2;
xvi. 11, 10, 9, 8, 7, 6, 5, 4, 3 and 1; or
xvii. 11, 10, 9, 8, 7, 6, 5, 4, 3, 2 and 1.

In embodiments, the introgressed sequence comprises one or more of SEQ ID NO: 5, SEQ ID NO: 10, SEQ ID NO: 15, SEQ ID NO: 20, SEQ ID NO: 25, SEQ ID NO: 30, SEQ ID NO: 35, SEQ ID NO: 40, SEQ ID NO: 45, SEQ ID NO: 50, and/or SEQ ID NO: 55, or a sequence that is at least 80%, 85%, 90%, 95%, 96%, 97%, 98%, 99% or more identical to one or more of the foregoing sequences and comprises the indicated Peronospora resistance-associated SNP marker genotype.

In embodiments, the plant according to the invention is a cultivated plant.

The plant can be heterozygous or homozygous for the introgressed sequence.

In embodiments, the introgressed sequence confers resistance against at least Peronospora effusa races Pe: 1-18.

In embodiments, the introgressed sequence is as comprised in S. oleracea line 21BN L002487 or S. oleracea line 21BNL002472, representative seed of S. oleracea line 21BNL002487 and S. oleracea line 21BNL002472 having been deposited with the NCIMB under Accession No. NCIMB 43893 and Accession No. NCIMB 44060, respectively.

In embodiments, the plants of the invention can be an inbred, a dihaploid or a hybrid plant. Generally, the plants of the invention are diploid.

As a further aspect, the invention provides a plant of Spinacia oleracea line 21BNL002487 or S. oleracea line 21BNL002472, and an F1 progeny plant thereof.

It is a further embodiment to provide a plant part, organ or tissue obtained from a spinach plant according to any of the embodiments described herein, including but not limited to leaves, stems, roots, flowers or flower parts, shoots, gametophytes, sporophytes, pollen, anthers, microspores, egg cells, zygotes, embryos, meristematic regions, callus tissue, seeds, cuttings, cell or tissue cultures or any other part or product of the plant, which comprise the introgressed sequence. In embodiments, the plant part, organ or tissue exhibits the Peronospora resistance according to the invention, particularly when grown into a spinach plant that produces spinach leaves.

Further provided is a seed that produces a plant according to the invention.

Still further is provided a seed produced by a plant according to the invention.

In another embodiment is considered the use of a spinach plant, plant part or seed according to any of the embodiments of the invention for producing and harvesting spinach leaves.

In another embodiment, the invention relates to the use of a spinach plant, plant part (e.g., leaves) or seed according to any embodiment described herein, to produce a further spinach plant, plant part or seed having the Peronospora resistance of the invention.

In another embodiment the invention relates to the use of a spinach plant, plant part or seed according to any embodiment of the invention, wherein the spinach plant, plant part or seed is Spinacia oleracea line 21BNL002487 or S. oleracea line 21BNL002472, or a progeny or an ancestor thereof.

As yet another aspect, the invention provides a method of producing spinach seed (e.g., S. oleracea seed), the method comprising growing a spinach plant from a seed according to the present invention, allowing the plant to produce further spinach seed, and optionally collecting the further spinach seed.

As still a further aspect, the invention provides a method for producing a spinach plant with enhanced resistance to Peronospora effusa (e.g., as compared with a control plant), the method comprising:
crossing a first S. oleracea plant according to the invention with a second spinach plant (e.g., an S. oleracea plant) lacking said introgressed sequence conferring resistance to Peronospora effusa to produce progeny plants; and
selecting a progeny plant comprising said introgressed sequence conferring resistance to Peronospora effusa.

In embodiments, said selecting step comprises detecting the presence of a resistance-associated genotype at one or more SNP markers in the chromosome interval defined by SNP marker 11 to 8, 7, 6, 5, 4 or 3 (inclusive). In embodiments, the method comprises detecting the presence of a resistance-associated genotype at one or more markers in the chromosome interval defined by SNP marker 11 to 2 or 1 (inclusive). In embodiments, the method comprises detecting the presence of a resistance-associated genotype at one or more markers in the chromosome interval defined by SNP marker 11 to 3 (inclusive). The method can comprise detecting a resistance-associated genotype at any combination of the markers in the chromosome interval, for example, as shown in Tables 3, 4 and 5.

Optionally said selecting step comprises detecting the presence of a resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45;
j) an A genotype in the heterozygous or homozygous state for SNP marker 10 in SEQ ID NO: 50; and/or
k) a G genotype in the heterozygous or homozygous state for SNP marker 11 in SEQ ID NO: 55,
thereby producing a plant with enhanced resistance to Peronospora effusa.

Optionally, the method further comprises:
selfing the selected progeny or crossing the selected progeny plant with another spinach plant (e.g., a S. oleracea plant) to produce further progeny. In embodiments, the further progeny are selected and selfed/crossed for 2 to 10 more generations.

As yet another aspect, the invention provides a method for producing a F1 hybrid spinach plant with enhanced resistance to Peronospora effusa (e.g., as compared with a control spinach plant), the method comprising crossing an inbred spinach plant (e.g., a S. oleracea plant), which is a plant according to any embodiment of the invention, with a different inbred spinach plant (e.g., S. oleracea plant) to produce a F1 hybrid progeny plant. In embodiments, the different inbred spinach plant does not comprise the introgressed sequence conferring enhanced resistance to Peronospora effusa, and is optionally susceptible to Peronospora effusa.

In representative embodiments, the method of producing a F1 hybrid spinach plant further comprises detecting the presence of a resistance-associated genotype at one or more SNP markers in the chromosome interval defined by SNP marker 11 to 8, 7, 6, 5, 4 or 3 (inclusive). In embodiments, the method comprises detecting the presence of a resistance-associated genotype at one or more markers in the chromosome interval defined by SNP marker 11 to 2 or 1 (inclusive). In embodiments, the method comprises detecting the presence of a resistance-associated genotype at one or more markers in the chromosome interval defined by SNP marker 11 to 3 (inclusive). The method can comprise detecting a resistance-associated genotype at any combination of the markers in the chromosome interval, for example, the non-exhaustive listing of SNP markers as shown in Tables 3, 4 and 5.

Optionally, the method comprises detecting the presence in the F1 hybrid progeny plant of a resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45;
j) an A genotype in the heterozygous or homozygous state for SNP marker 10 in SEQ ID NO: 50; and/or
k) a G genotype in the heterozygous or homozygous state for SNP marker 11 in SEQ ID NO: 55; and
optionally selecting the plant based on the presence of the genotype at the one or more SNP markers.

In yet another aspect, the invention provides a method for identifying a spinach plant (e.g., a S. oleracea plant) with enhanced resistance to Peronospora effusa (e.g., as compared with a control spinach plant), said method comprising the step of detecting the presence of a resistance-associated genotype at one or more SNP markers in the chromosome interval defined by SNP marker 11 to 8, 7, 6, 5, 4 or 3 (inclusive). In embodiments, the method comprises detecting the presence of a resistance-associated genotype at one or more markers in the chromosome interval defined by SNP marker 11 to 2 or 1 (inclusive). In embodiments, the method comprises detecting the presence of a resistance-associated genotype at one or more markers in the chromosome interval defined by SNP marker 11 to 3 (inclusive). The method can comprise detecting any combination of markers in the chromosome interval, for example, the non-exhaustive listing of SNP markers as shown in Tables 3, 4 and 5.

In embodiments, the method comprises detecting the presence of a resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45;
j) an A genotype in the heterozygous or homozygous state for SNP marker 10 in SEQ ID NO: 50; and/or
k) a G genotype in the heterozygous or homozygous state for SNP marker 11 in SEQ ID NO: 55,
thereby identifying a spinach plant with enhanced resistance to Peronospora effusa.

In embodiments, said method further comprises selecting a spinach plant comprising said resistance-associated genotype at the one or more SNP markers, and crossing the selected spinach plant with a second spinach plant (e.g., a S. oleracea plant) to produce a progeny spinach plant that comprises said resistance-associated genotype at the one or more SNP markers and has enhanced resistance to Peronospora effusa.

In another embodiment the invention relates to a method of providing a spinach plant, plant part (e.g., leaf) or seed with enhanced resistance to Peronospora, wherein said method comprises the following steps:
crossing a first spinach plant (e.g., S. oleracea plant) according to any embodiment of the invention with a second spinach plant (e.g., a S. oleracea plant) lacking the Peronospora resistance-conferring introgressed sequence of the invention;
obtaining a progeny spinach plant; and,
optionally, selecting a plant of said progeny characterized in that said progeny plant exhibits enhanced resistance to Peronospora effusa.

In embodiments, said selecting step comprises detecting the presence of a resistance-associated genotype at one or more SNP markers in the chromosome interval defined by SNP marker 11 to 8, 7, 6, 5, 4 or 3 (inclusive). In embodiments, the selecting step comprises detecting the presence of a resistance-associated genotype at one or more markers in the chromosome interval defined by SNP marker 11 to 2 or 1 (inclusive). In embodiments, the selecting step comprises detecting the presence of a resistance-associated genotype at one or more markers in the chromosome interval defined by SNP marker 11 to 3 (inclusive). The method can comprise detecting a resistance-associated genotype at any combination of the markers in the chromosome interval, for example, the non-exhaustive listing of SNP markers as shown in Tables 3, 4 and 5.

In embodiments, said selecting step comprises detecting the presence of the resistance-associated genotype at one or more SNP markers as set forth in a) to k) above, in any combination.

According to the preceding embodiment, optionally, the first spinach plant is a plant of Spinacia oleracea line 21BNL002487 or S. oleracea line 21BNL002472, or a progeny or an ancestor thereof.

In another embodiment the invention relates to a method of identifying a spinach plant (e.g., a S. oleracea plant) comprising a Peronospora resistance-conferring introgressed sequence of the invention, wherein said method comprises the steps of:
a) providing a spinach (e.g., S. oleracea) population segregating for the Peronospora resistance trait;
b) screening the segregating population for a plant exhibiting resistance to Peronospora, wherein said resistance trait can be identified by the presence of a Peronospora resistance-conferring introgressed sequence of the invention; and
c) selecting a plant from the segregating population, wherein said plant comprises the Peronospora resistance trait.

Optionally, identifying the presence of the introgressed sequence of the invention comprises detecting the presence of a resistance-associated genotype at one or more SNP markers in the chromosome interval defined by SNP marker 11 to 8, 7, 6, 5, 4 or 3 (inclusive). In embodiments, the method comprises detecting the presence of a resistance-associated genotype at one or more markers in the chromosome interval defined by SNP marker 11 to 2 or 1 (inclusive). In embodiments, the method comprises detecting the presence of a resistance-associated genotype at one or more markers in the chromosome interval defined by SNP marker 11 to 3 (inclusive). The method can comprise detecting a resistance-associated genotype at any combination of the markers in the chromosome interval, for example, the non-exhaustive listing of SNP markers as shown in Tables 3, 4 and 5.

In embodiments, identifying the presence of the introgressed sequence of the invention comprises detecting the presence of the resistance-associated genotype at one or more SNP markers as set forth in a) to k) above, in any combination.

In embodiments, the method comprises detecting the presence of the Peronospora resistance-associated genotype at two or more, three or more, four or more, five or more, six or more, seven or more, or eight or more of the SNP markers of c) to k) in any combination, optionally at all of the markers of c) to k).

In embodiments, the method comprises detecting the presence of the Peronospora resistance-associated genotype at two or more, three or more, four or more, five or more, six or more, seven or more, eight or more, nine or more, or ten or more of the SNP markers of a) to k) in any combination, optionally at all of the markers of a) to k).

In representative embodiments, the method comprises detecting the presence of the Peronospora resistance-associated genotype at any of the following combinations of SNP markers:
i. 11 and 1;
ii. 11 and 2;
iii. 11 and 3;
iv. 11 and 4;
v. 11 and 5;
vi. 11 and 6;
vii. 11 and 7;
viii. 11 and 8;
ix. 11, 10, 9 and 8;
x. 11, 10, 9, 8 and 7;
xi. 11, 10, 9, 8, 7 and 6;
xii. 11, 10, 9, 8, 7, 6 and 5;
xiii. 11, 10, 9, 8, 7, 6, 5 and 4;
xiv. 11, 10, 9, 8, 7, 6, 5, 4 and 3;
xv. 11, 10, 9, 8, 7, 6, 5, 4, 3 and 2;
xvi. 11, 10, 9, 8, 7, 6, 5, 4, 3 and 1; or
xvii. 11, 10, 9, 8, 7, 6, 5, 4, 3, 2 and 1.

In embodiments, the method comprises detecting the presence of one or more of SEQ ID NO: 5, SEQ ID NO: 10, SEQ ID NO: 15, SEQ ID NO: 20, SEQ ID NO: 25, SEQ ID NO: 30, SEQ ID NO: 35, SEQ ID NO: 40, SEQ ID NO: 45, SEQ ID NO: 50, and/or SEQ ID NO: 55, or a sequence that is at least 80%, 85%, 90%, 95%, 96%, 97%, 98%, 99% or more identical to one or more of the foregoing sequences and comprises the indicated Peronospora resistance-associated SNP marker genotype.

In embodiments, the spinach plant that is produced, selected or identified has resistance against at least Peronospora effusa races Pe: 1-18.

In further representative embodiments:
the A genotype for SNP marker 1 can be identified in a PCR by amplification of a nucleic acid fragment with a pair of oligonucleotide primers: forward primer of SEQ ID NO: 1 and reverse primer of SEQ ID NO: 4, and probe of SEQ ID NO: 2;
the A genotype for SNP marker 2 can be identified in a PCR by amplification of a nucleic acid fragment with a pair of oligonucleotide primers: forward primer of SEQ ID NO: 6 and reverse primer of SEQ ID NO: 9, and probe of SEQ ID NO: 7;
the A genotype for SNP marker 3 can be identified in a PCR by amplification of a nucleic acid fragment with a pair of oligonucleotide primers: forward primer of SEQ ID NO: 11 and reverse primer of SEQ ID NO: 14, and probe of SEQ ID NO: 12;
the G genotype for SNP marker 4 can be identified in a PCR by amplification of a nucleic acid fragment with a pair of oligonucleotide primers: forward primer of SEQ ID NO: 16 and reverse primer of SEQ ID NO: 19, and probe of SEQ ID NO: 17;
the C genotype for SNP marker 5 can be identified in a PCR by amplification of a nucleic acid fragment with a pair of oligonucleotide primers: forward primer of SEQ ID NO: 21 and reverse primer of SEQ ID NO: 24, and probe of SEQ ID NO: 22;
the A genotype for SNP marker 6 can be identified in a PCR by amplification of a nucleic acid fragment with a pair of oligonucleotide primers: forward primer of SEQ ID NO: 26 and reverse primer of SEQ ID NO: 29, and probe of SEQ ID NO: 27;
the T genotype for SNP marker 7 can be identified in a PCR by amplification of a nucleic acid fragment with a pair of oligonucleotide primers: forward primer of SEQ ID NO: 31 and reverse primer of SEQ ID NO: 34, and probe of SEQ ID NO: 32;
the A genotype for SNP marker 8 can be identified in a PCR by amplification of a nucleic acid fragment with a pair of oligonucleotide primers: forward primer of SEQ ID NO: 36 and reverse primer of SEQ ID NO: 39, and probe of SEQ ID NO: 37;
the G genotype for SNP marker 9 can be identified in a PCR by amplification of a nucleic acid fragment with a pair of oligonucleotide primers: forward primer of SEQ ID NO: 41 and reverse primer of SEQ ID NO: 44, and probe of SEQ ID NO: 42;
the A genotype for SNP marker 10 can be identified in a PCR by amplification of a nucleic acid fragment with a pair of oligonucleotide primers: forward primer of SEQ ID NO: 46 and reverse primer of SEQ ID NO: 49, and probe of SEQ ID NO: 47; and
the G genotype for SNP marker 11 can be identified in a PCR by amplification of a nucleic acid fragment with a pair of oligonucleotide primers: forward primer of SEQ ID NO: 51 and reverse primer of SEQ ID NO: 54, and probe of SEQ ID NO: 52.

These and other aspects of the invention are described in more detail in the description of the invention that follows.

### DETAILED DESCRIPTION OF THE INVENTION

This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

All publications, patent applications, patents and other references cited herein are incorporated by reference in their entireties for the teachings relevant to the sentence and/or paragraph in which the reference is presented.

Nucleotide sequences provided herein are presented in the 5′ to 3′ direction, from left to right and are presented using the standard code for representing nucleotide bases as set forth in 37 CFR §§ 1.821-1.825 and the World Intellectual Property Organization (WIPO) Standard ST.25, for example: adenine (A), cytosine (C), thymine (T), and guanine (G).

Amino acids are likewise indicated using the WIPO Standard ST.25, for example: alanine (Ala; A), arginine (Arg; R), asparagine (Asn; N), aspartic acid (Asp; D), cysteine (Cys; C), glutamine (Gln; Q), glutamic acid (Glu; E), glycine (Gly; G), histidine (His; H), isoleucine (Ile; 1), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

### Definitions

The technical terms and expressions used within the scope of this application are generally to be given the meaning commonly applied to them in the pertinent art of plant breeding if not otherwise indicated herein below.

As used in this specification and the appended claims, the singular forms “a”, “an”, and “the” include plural referents unless the context clearly dictates otherwise. Thus, for example, reference to “a plant” includes one or more plants.

As used herein, the term “about” when referring to a value or to an amount of mass, weight, time, volume, concentration or percentage, and the like is meant to encompass variations of in some embodiments ±20%, in some embodiments ±10%, in some embodiments ±5%, in some embodiments ±1%, in some embodiments ±0.5%, and in some embodiments ±0.1% from the specified amount.

As used herein, a “spinach” plant refers to any plant in the genus Spinacia, including but not limited to the cultivated species S. oleracea and wild spinach species S. turkestanica and S. tetrandra. In embodiments, the spinach plant is a S. oleracea. In embodiments, the spinach plant is cultivated.

A “cultivated spinach” plant is understood within the scope of the invention to refer to a plant that is no longer in the natural state but has been developed and domesticated by human care and for agricultural use and/or human consumption, and excludes wild spinach species and accessions, such as S. turkestanica accession CGN080741. In embodiments, the cultivated spinach plant is a hybrid plant. Alternatively or additionally, the cultivated spinach plant is a S. oleracea plant. In the context of an interspecific cross between a S. oleracea plant and a wild spinach, a progeny plant of said interspecific cross is considered a “cultivated spinach” plant when said progeny plant has been backcrossed at least two times against a S. oleracea plant.

An “allele” is understood within the scope of the invention to refer to alternative or variant forms of a gene or other genetic element. Such alternative or variant forms may be the result of single nucleotide polymorphisms, insertions, inversions, translocations or deletions, or the consequence of gene regulation caused, for example, by chemical or structural modification, transcription regulation or post-translational modification/regulation. In a diploid cell or organism, the two alleles of a given gene or genetic element typically occupy corresponding loci on a pair of homologous chromosomes.

“Peronospora effusa” is an Oomycete that causes downy mildew in spinach. As used herein, the terms Peronospora effusa and Peronospora are used interchangeably, unless the context indicates otherwise.

As used herein, the terms “resistance” and “resistant” (and like terms) refer to the ability of a plant to restrict the growth and development of a specified pathogen and/or the damage caused by the pathogen when compared to susceptible plants under similar environmental conditions and pathogen pressure. Resistance can be qualitative or quantitative. In embodiments, a “resistant” plant exhibits reduced, essentially no, or even no symptoms to a specific pathogen. In some embodiments, “resistant” plants show some symptoms but are still able to produce marketable product with an acceptable yield, e.g., the yield may be reduced and/or the plants may be stunted as compared with the yield and/or growth in the absence of the pathogen. In embodiments, a spinach plant according to the invention is resistant to at least Peronospora effusa races Pe: 1-18 as characterized and classified according to IWGP (International Working Group on Peronospora). In embodiments, a Peronospora resistant spinach plant of the invention exhibits no or very few necroses with no or very sparse sporulation under standard test conditions, for example, the test conditions defined in Example 1 below, e.g., when inoculated with any of Peronospora races Pe: 1-18. In embodiments, the Peronospora resistance of the invention is dominant. In embodiments, the Peronospora resistance is qualitative. In embodiments, the Peronospora resistance of the invention exhibits monogenic inheritance.

The term “enhanced Peronospora resistance” (and like terms) is herein understood to mean that a plant is more resistant to at least one Peronospora race or isolate (e.g. a statistically significant improvement in Peronospora resistance as compared with a control spinach plant not comprising an introgressed sequence of the invention, for example, at p<0.1, p<0.05 or p<0.01 using Student's test) and/or has a broader spectrum of Peronospora resistance (e.g., has resistance against one or more additional Peronospora races/isolates) as compared with a control spinach plant not comprising an introgressed sequence of the invention. In embodiments, “enhanced Peronospora resistance” refers to the provision of an additional resistance against one or more Peronospora races or isolates against which the plant already has resistance (i.e., stacking of resistance), thereby lowering the risk of the Peronospora resistance being broken as compared with a plant lacking said introgressed sequence (i.e., as a form of resistance management).

A “control spinach plant” is understood within the scope of the invention to mean a spinach plant of the same species as the spinach plant of the invention (e.g., S. oleracea), but the control spinach plant does not have the introgressed sequence of the present invention conferring Peronospora resistance. In embodiments, the control spinach plant has the same genetic background as the spinach plant of the present invention, and optionally is a plant belonging to the same plant variety, but does not comprise the introgressed sequence of the present invention. Plant variety is herein understood according to the definition of the International Union for the Protection of New Varieties of Plants (UPOV). Thus, according to this embodiment, a control spinach plant may be a near-isogenic line, an inbred line or a hybrid provided that the control spinach plant has the same genetic background as the spinach plant of the present invention except the control plant does not have the introgressed sequence of the present invention conferring Peronospora resistance. Generally, the control spinach plant is grown for the same length of time and under the same environmental conditions as the plant of the invention.

The term “trait” refers to a characteristic or a phenotype (e.g., a disease resistance such as Peronospora resistance). A trait may be inherited in a dominant or recessive manner, or in a partial or incomplete-dominant manner. In the context of the present invention, the Peronospora resistance of the present invention is a dominant trait. A spinach plant of the invention can therefore be homozygous or heterozygous for the trait. Furthermore, a trait may be inherited in a monogenic or polygenic manner, or may result from the interaction of one or more genes with the environment. In the context of the present invention, the Peronospora resistance-conferring introgressed sequence located on chromosome 3 exhibits monogenic inheritance, and is sufficient to confer the Peronospora resistance trait.

The terms “hybrid”, “hybrid plant”, and “hybrid progeny” refer to an individual produced from genetically different parents (e.g., a genetically heterozygous or mostly heterozygous individual), for example, the result of a cross of two inbred lines.

The term “inbred line” refers to a genetically homozygous or nearly homozygous population. An inbred line, for example, can be derived through several cycles of brother/sister breeding or of selfing or by dihaploid production.

The term “dihaploid line” refers to stable inbred lines produced from anther culture. Some pollen grains (haploid) when cultivated on specific medium and under specific conditions can develop plantlets containing n chromosomes. The chromosomes of these haploid plantlets are then “doubled” to produce a diploid (2n) plant. The progeny of these plantlets are named “dihaploid” and are essentially no longer segregating (i.e., are stable).

The term “cultivar” or “variety” refers to a horticulturally created variety, as distinguished from a naturally occurring variety. In some embodiments of the present invention the cultivars or varieties are commercially valuable.

The term “genetically fixed” refers to a genetic element that has been stably incorporated into the genome of a plant that normally does not contain the genetic element. When genetically fixed, the genetic element can be transmitted in an easy and predictable manner to other plants by sexual crosses.

The term “plant” or “plant part’ refers herein to a plant part, organ or tissue obtainable from a plant according to the invention, including but not limited to leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures (including callus cultures) or any other part or product of the plant. In embodiments, the plant part comprises the introgressed sequence of the invention. In embodiments, the plant part exhibits the Peronospora resistance trait according to the invention, particularly when grown into a plant that produces spinach leaves.

A “plant” is a plant at any stage of development.

A plant “seed” is a seed that grows into a plant according to any of the embodiments of the invention.

A “plant cell” is a structural and physiological unit of a plant, comprising a protoplast and a cell wall. The plant cell may be in form of an isolated single cell or a cultured cell, or as a part of higher organized unit such as, for example, plant tissue, a plant organ, or a whole plant.

“Plant cell culture” means cultures of plant units such as, for example, protoplasts, cells, cells in plant tissues, pollen, pollen tubes, ovules, embryo sacs, zygotes and embryos at various stages of development.

A “plant organ” is a distinct and visibly structured and differentiated part of a plant such as a root, stem, leaf, flower bud, or embryo.

“Plant tissue” as used herein means a group of plant cells organized into a structural and functional unit. Any tissue of a plant in planta or in culture is included. This term includes, but is not limited to, whole plants, plant organs, plant seeds, tissue culture and any groups of plant cells organized into structural and/or functional units. The use of this term in conjunction with, or in the absence of, any specific type of plant tissue as listed above or otherwise embraced by this definition is not intended to be exclusive of any other type of plant tissue.

“Processed food” is understood within the scope of the invention to mean a food that has been altered from its natural state. Methods used for processing food include but are not limited to cutting, slicing, dicing, grinding, canning, freezing, refrigeration, dehydration, heating and aseptic processing, and combinations thereof.

“Fresh cut market” is understood within the scope of the invention to mean vegetables on the market that have been minimally processed.

As used herein, the term “marker allele” or “allele of a marker locus” (and similar terms) refers to an allele (as defined herein) at a polymorphic locus that is used as a marker to locate and/or indicate the presence of one or more genetically linked loci that contribute to variability of a phenotypic trait(s) (e.g., a Peronospora resistance locus).

As used herein, “marker locus” refers to a region on a chromosome that comprises a nucleotide or a polynucleotide sequence that is present in an individual's genome and that is genetically linked with one or more loci of interest, which may comprise a gene or any other genetic determinant or factor contributing to a trait. “Marker locus” also refers to a region on a chromosome that comprises a polynucleotide sequence complementary to a genomic sequence, such as a sequence of a nucleic acid used as a probe. A marker locus can be used to track the presence of a second linked locus, e.g., a linked locus that encodes or contributes to expression of a phenotypic trait. For example, a marker locus can be used to monitor segregation of alleles at a locus, such as a quantitative trait locus (QTL) or a gene, that are genetically or physically linked to the marker locus.

As used herein, the term “breeding”, and grammatical variants thereof, refers to any process that generates a progeny individual. Breeding can be sexual or asexual, or any combination thereof. Exemplary non-limiting types of breeding include crossings, selfing, doubled haploid generation, and combinations thereof.

As used herein, the phrase “established breeding population” refers to a collection of potential breeding partners produced by and/or used as parents in a breeding program; e.g., a commercial breeding program. The members of the established breeding population are typically well-characterized genetically and/or phenotypically. For example, several phenotypic traits of interest might have been evaluated, e.g., under different environmental conditions, at multiple locations, and/or at different times. Alternatively or in addition, one or more genetic loci associated with expression of the phenotypic traits might have been identified and one or more of the members of the breeding population might have been genotyped with respect to the one or more genetic loci as well as with respect to one or more genetic markers that are associated with the one or more genetic loci.

As used herein, the term “diploid” plant refers to a plant that has two sets of chromosomes, typically one from each of its two parents. However, it is understood that in some embodiments a diploid plant can receive its “maternal” and “paternal” sets of chromosomes from the same single organism, such as when a plant is selfed to produce a subsequent generation of plants. In embodiments, the plants (and parts thereof) of the present invention are diploid plants.

“Homozygous” is understood within the scope of the invention to refer to like alleles at one or more corresponding loci on homologous chromosomes.

“Heterozygous” is understood within the scope of the invention to refer to unlike alleles at one or more corresponding loci on homologous chromosomes.

A “dominant” allele is understood within the scope of the invention to refer to an allele that determines the phenotype when present in the heterozygous or homozygous state.

A “recessive” allele refers to an allele that determines the phenotype only when present in the homozygous state.

“Backcrossing” is understood within the scope of the invention to refer to a process in which a hybrid progeny is repeatedly crossed back to one of the parents (the “recurrent” parent). Different recurrent parents may be used in subsequent backcrosses.

“Locus” is understood within the scope of the invention to refer to a region on a chromosome, which comprises a gene or any other genetic element or factor contributing to a trait.

“Genetic linkage” is understood within the scope of the invention to refer to an association of two or more different genetic elements during inheritance due to location of genes in proximity on the same chromosome, measured by percent recombination between loci (centi-Morgan, cM).

For the purpose of the present invention, the term “co-segregation” refers to the situation in which the allele for the trait and the allele(s) for the marker(s) tend to be transmitted together because they are physically close together on the same chromosome (i.e., reduced recombination between them because of their physical proximity) resulting in a non-random association of their alleles as a result of their proximity on the same chromosome. The term “associated with” can be used with an equal meaning.

As used herein, the term “genetic architecture at the quantitative/qualitative trait locus” refers to a genomic region which is statistically correlated to the phenotypic trait of interest and represents the underlying genetic basis of the phenotypic trait of interest.

As used herein, the phrase “genetic marker” or “molecular marker” refer to a feature of an individual's genome (e.g., a nucleotide or a polynucleotide sequence that is present in an individual's genome) that is associated with one or more loci of interest. In some embodiments, a genetic marker is polymorphic in a population of interest, or the locus occupied by the polymorphism, depending on context. Genetic/molecular markers include, for example, single nucleotide polymorphisms (SNPs), indels (i.e., insertions/deletions), simple sequence repeats (SSRs), restriction fragment length polymorphisms (RFLPs), random amplified polymorphic DNAs (RAPDs), cleaved amplified polymorphic sequence (CAPS) markers, Diversity Arrays Technology (DArT) markers, and amplified fragment length polymorphisms (AFLPs), among many other examples. Genetic/molecular markers can, for example, be used to locate genetic loci containing alleles on a chromosome that contribute to variability of a phenotypic trait(s). The phrase “genetic marker” or “molecular marker” can also refer to a polynucleotide sequence complementary to a genomic sequence, such as a sequence of a nucleic acid used as a probe. In embodiments, a genetic/molecular marker can be physically located in a position on a chromosome that is within or outside the genetic locus with which it is associated (i.e., is intragenic or extragenic, respectively).

As used herein, the term “genotype” refers to the genetic constitution of a cell or organism. An individual's genotype for a set of genetic markers includes the specific alleles for one or more genetic marker loci present in the individual's haplotype. As is known in the art, a genotype can relate to a single locus or to multiple loci, whether the loci are related or unrelated and/or are linked or unlinked. In some embodiments, an individual's genotype relates to one or more genes that are related in that the one or more genes are involved in the expression of a phenotype of interest (e.g., a quantitative trait as defined herein). Thus, in some embodiments a genotype comprises one or more alleles present within an individual at one or more genetic loci. In some embodiments, a genotype is expressed in terms of a haplotype (also as defined herein).

As used herein, the term “germplasm” refers to the totality of the genotypes of a population or other group of individuals (e.g., a species). The term “germplasm” can also refer to plant material; e.g., a group of plants that act as a repository for various alleles. The phrase “adapted germplasm” refers to plant materials of proven genetic superiority, e.g., for a given environment or geographical area, while the phrases “non-adapted germplasm,” “raw germplasm,” and “exotic germplasm” refer to plant materials of unknown or unproven genetic value, e.g., for a given environment or geographical area. As such, the phrase “non-adapted germplasm” refers in some embodiments to plant materials that are not part of an established breeding population and that do not have a known relationship to a member of the established breeding population.

A “haplotype” is the genotype of an individual at a plurality of genetic loci, i.e., a combination of alleles. Typically, the genetic loci that define a haplotype are physically and genetically linked, i.e., multiple loci along the same chromosome segment.

As used herein, the terms “introgression,” “introgressing” and “introgressed” (and grammatical variants thereof) refer to both the natural and artificial transmission of a desired allele or combination of desired alleles of a genetic locus or genetic loci from one genetic background to another. For example, a desired allele at a specified locus can be transmitted to at least one progeny via a sexual cross between two parents, where at least one of the parents has the desired allele in its genome (the “donor” parent). Alternatively, for example, transmission of an allele can occur by recombination between two donor genomes, e.g., in a fused protoplast, where at least one of the donor protoplasts has the desired allele in its genome. Offspring comprising the desired allele can be repeatedly backcrossed to a recurrent parent to create a line having a desired genetic background and selected for the desired allele, with the result being that the desired allele becomes fixed in the desired genetic background. For example, a marker associated with enhanced Peronospora resistance may be introgressed from a donor parent into a recurrent parent that does not comprise the introgressed sequence. The resulting offspring can optionally be repeatedly backcrossed to the recurrent parent and selected until the progeny possess the introgressed sequence conferring Peronospora resistance in the recurrent parent background.

As used herein, the term “linkage”, and grammatical variants thereof, refers to the tendency of alleles at different loci on the same chromosome to segregate together more often than would be expected by chance if their transmission were independent, in some embodiments as a consequence of their physical proximity.

As used herein, the phrase “nucleic acid” refers to any physical string of monomer units that can be corresponded to a string of nucleotides, including a polymer of nucleotides (e.g., a typical DNA, cDNA or RNA polymer), modified oligonucleotides (e.g., oligonucleotides comprising bases that are not typical to biological RNA or DNA, such as 2′-O-methylated oligonucleotides), and the like. In some embodiments, a nucleic acid can be single-stranded, double-stranded, multi-stranded, or combinations thereof. Unless otherwise indicated, a particular nucleic acid sequence of the presently disclosed subject matter optionally comprises and/or encodes complementary sequences, in addition to any sequence explicitly indicated (i.e., is double-stranded).

As used herein, the term “plurality” refers to more than one. Thus, a “plurality of individuals” refers to at least two individuals. In some embodiments, the term plurality refers to more than half of the whole. For example, in some embodiments a “plurality of a population” refers to more than half the members of that population. These usages of the term “plurality” will be apparent to those skilled in the art depending on context.

As used herein, the term “progeny” refers to the descendant(s) of a particular cross (including selfings). Typically, progeny result from breeding of two individuals, although some species (particularly some plants and hermaphroditic animals) can be selfed (i.e., the same plant acts as the donor of both male and female gametes). The descendant(s) can be of any generation, for example, of the F₁, the F₂, or any subsequent generation.

As used herein, the phrase “qualitative trait” refers to a phenotypic trait that is controlled by one or a few genes that exhibit major phenotypic effects. Because of this, qualitative traits are typically simply inherited. Examples in plants include, but are not limited to, flower colour, and several known disease resistances such as, for example, the Peronospora resistance of the present invention.

As used herein, the phrase “quantitative trait” refers to a phenotypic trait that can be described numerically (i.e., quantitated or quantified). A quantitative trait typically exhibits continuous variation between individuals of a population; that is, differences in the numerical value of the phenotypic trait are slight and grade into each other. Oftentimes, the frequency distribution in a population of a quantitative phenotypic trait exhibits a bell-shaped curve (i.e., exhibits a normal distribution between two extremes). A “quantitative trait” is typically the result of a genetic locus interacting with the environment or of multiple genetic loci interacting with each other and/or with the environment. Examples of quantitative traits include plant height and yield.

As used herein, the terms “quantitative trait locus” (QTL) and “marker trait association” refer to an association between a genetic marker and a chromosomal region and/or gene that affects the phenotype of a trait of interest. Typically, this is determined statistically, e.g., based on one or more methods published in the literature.

A quantitative trait locus or qualitative trait locus can be a chromosomal region and/or a genetic locus with at least two alleles that differentially affect a phenotypic trait.

The term “recipient plant” is used herein to indicate a plant that is to receive DNA obtained from a donor plant that comprises an introgressed sequence for a trait of interest (e.g., Peronospora resistance). Said “recipient plant” may or may not already comprise one or more native or introgressed sequences for Peronospora resistance, in which case the term indicates a plant that is to receive an additional introgressed sequence.

The term “natural genetic background” is used herein to indicate the original genetic background of a genetic sequence of interest. Such a genetic background may for instance be the genome of a wild accession. Conversely, a method that involves the transfer of DNA, via e.g. breeding, comprising transferring the genetic sequence from e.g. a wild spinach species to another spinach species (e.g., S. oleracea), will result in this genetic sequence not being in its natural genetic background.

A “donor plant” is understood within the scope of the invention to mean the plant that provides at least one sequence for enhanced Peronospora resistance (i.e., to be introgressed into the recipient plant).

“Marker-based selection” is understood within the scope of the invention to refer to, e.g., the use of genetic markers to detect one or more nucleic acids from the plant, where the nucleic acid is associated with a desired trait to identify plants that carry an allele(s) conferring desirable (or undesirable) traits, so that those plants can be used (or avoided) in a selective breeding program.

A single nucleotide polymorphism (SNP), a variation at a single site in DNA, is the most frequent type of variation in the genome. A SNP is a DNA sequence variation occurring when a single nucleotide—A, T, C, or G—in the genome (or other shared sequence) differs between members of a biological species or paired chromosomes in an individual. For example, two sequenced DNA fragments from different individuals, AAGCCTA to AAGCTTA, contain a difference in a single nucleotide. In this case there are two alleles: C and T. The basic principles of SNP array are the same as the DNA microarray. The three components of the SNP arrays are: the array that contains nucleic acid sequences (e.g., amplified sequence or target), one or more labelled allele-specific oligonucleotide probes, and a detection system that records and interprets the hybridization signal. The presence or absence of the desired allele may be determined, e.g., by real-time PCR using double-stranded DNA dyes or the fluorescent reporter probe method.

“PCR (Polymerase chain reaction)” is understood within the scope of the invention to refer to a method of producing relatively large amounts of specific regions of DNA or subset(s) of the genome, thereby making possible various analyses that are based on those regions.

“PCR primer” is understood within the scope of the invention to refer to relatively short fragments of single-stranded DNA used in the PCR amplification of specific regions of DNA.

“Phenotype” is understood within the scope of the invention to refer to a distinguishable characteristic(s) of a genetically controlled trait.

As used herein, the phrase “phenotypic trait” refers to the appearance or other detectable characteristic of an individual, resulting from the interaction of its genome, proteome and/or metabolome with the environment.

“Polymorphism” is understood within the scope of the invention to refer to the presence in a population of two or more different forms of a gene, genetic marker, or inherited trait or a gene product obtainable, for example, through alternative splicing, alternative start or stop codons, DNA methylation, etc.

“Probe” as used herein refers to a group of atoms or molecules that is capable of recognising and binding to a specific target molecule or cellular structure and thus allowing detection of the target molecule or structure. In embodiments, a “probe” refers to a labelled DNA or RNA sequence that can be used to detect the presence of and, optionally, to quantitate a complementary sequence by molecular hybridization.

As used herein, the phrases “sexually crossed” and “sexual reproduction” in the context of the presently disclosed subject matter refers to the fusion of gametes to produce progeny (e.g., by fertilization, such as to produce seed by pollination in plants). A “sexual cross” or “cross-fertilization” is in some embodiments fertilization of one individual by another (e.g., cross-pollination in plants).

“Selective breeding” is understood within the scope of the invention to refer to a program of breeding that uses plants that possess or display desirable traits as parents.

The term “selfing” refers in some embodiments to the production of seed by self-fertilization or self-pollination; i.e., pollen and ovule are from the same plant.

The term “hybridize” as used herein refers to conventional hybridization conditions, for example, hybridization conditions at which 5×SSPE, 1% SDS, 1×Denhardts solution is used as a solution and/or hybridization temperatures are between 35° C. and 70° C., for example, 65° C. After hybridization, washing is optionally carried out first with 2×SSC, 1% SDS and subsequently with 0.2×SSC at temperatures between 35° C. and 75° C., particularly between 45° C. and 65° C., but especially at 59° C. (regarding the definition of SSPE, SSC and Denhardts solution see Sambrook et al. Molecular Cloning: a Laboratory Manual. Cold Spring Harbor, N.Y.: Cold Spring Harbor Laboratory Press, 2001). High stringency hybridization conditions as for instance described in Sambrook et al, supra, can be used. In particular embodiments, stringent hybridization conditions are for instance present if hybridization and washing occur at 65° C. as indicated above. Non-stringent hybridization conditions for instance with hybridization and washing carried out at 45° C. are less preferred and at 35° C. even less.

In accordance with the present invention, the term “said position corresponding to position X”, X being any number to be found in the respective context in the present application, does not only include the respective position in the SEQ ID NO referred to afterwards but also includes any sequence encoding a genetic sequence of the invention, where, after alignment with the reference SEQ ID NO, the respective position might have a different number but corresponds to that indicated for the reference SEQ ID NO. Alignment of genetic sequences can be effected by applying various alignment tools known to the person skilled in the art.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes part I chapter 2 “Overview of principles of hybridization and the strategy of nucleic acid probe assays” Elsevier, New York. Generally, highly stringent hybridization and wash conditions are selected to be about 5° C. lower than the thermal melting point for the specific sequence at a defined ionic strength and pH. Typically, under “stringent conditions” a probe will hybridize to its target subsequence, but to no other sequences.

The “thermal melting point” is the temperature (under defined ionic strength and pH) at which 50% of the target sequence hybridizes to a perfectly matched probe. Very stringent conditions are selected to be equal to the melting temperature (Tₘ) for a particular probe. An example of stringent hybridization conditions for hybridization of complementary nucleic acids which have more than 100 complementary residues on a filter in a Southern or northern blot is 50% formamide with 1 mg of heparin at 42° C., with the hybridization being carried out overnight. An example of highly stringent wash conditions is 0.1 5M NaCl at 72° C. for about 15 minutes. An example of stringent wash conditions is a 0.2 times SSC wash at 65° C. for 15 minutes (see, Sambrook et al, supra, for a description of SSC buffer). Often, a high stringency wash is preceded by a low stringency wash to remove background probe signal. An example medium stringency wash for a duplex of, e.g., more than 100 nucleotides, is 1 times SSC at 45° C. for 15 minutes. An example low stringency wash for a duplex of, e.g., more than 100 nucleotides, is 4-6 times SSC at 40° C. for 15 minutes. For short probes (e.g., about 10 to 50 nucleotides), stringent conditions typically involve salt concentrations of less than about 1.0M Na ion, typically about 0.01 to 1.0 M Na ion concentration (or other salts) at pH 7.0 to 8.3, and the temperature is typically at least about 30° C. Stringent conditions can also be achieved with the addition of destabilizing agents such as formamide. In general, a signal to noise ratio of 2 times (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g. when a copy of a nucleic acid is created using the maximum codon degeneracy permitted by the genetic code.

### Plants, Seeds, Products.

In a first embodiment, the invention provides a spinach plant (e.g., a Spinacia oleracea plant) with enhanced resistance to Peronospora effusa comprising an introgressed sequence that confers a qualitative and dominant resistance to Peronospora effusa, wherein said introgressed sequence is located on chromosome 3. In embodiments, the plant comprises a G genotype in the heterozygous or homozygous state for SNP marker 11 in SEQ ID NO: 55 (i.e., the resistance-associate genotype/allele); wherein the resistance of the plant to Peronospora effusa is enhanced as compared with a S. oleracea plant lacking said introgressed sequence (e.g., a control plant). In embodiments, the resistance is a qualitative resistance. In embodiments, the resistance is a dominant resistance. In embodiments, the resistance exhibits monogenic inheritance. In embodiments, the introgressed sequence is comprised in S. oleracea line 21BNL002487 or S. oleracea line 21BNL002472, representative seed of S. oleracea 21 BNL002487 having been deposited with the NCIMB under Accession No. NCIMB 43893 and NCIMB Accession No. NCIMB 44060, respectively.

In embodiments, the plant of the invention is a cultivated plant, optionally a cultivated S. oleracea plant.

In embodiments, said introgressed sequence confers resistance against at least Peronospora effusa races Pe: 1-18.

In embodiments, the plant has no adverse agronomic phenotypes, for example, the plant does not exhibit dwarfism.

In embodiments, the introgressed sequence comprises one or more, two or more, three or more, four or more, five or more, six or more, seven or more, eight or more, nine or more, or ten or more of the SNP markers shown in Table 3, each having the resistance genotype as shown in Tables 3 to 5 (each SNP marker allele can optionally be present in a heterozygous or homozygous state), in any combination. Optionally, the introgressed sequence comprises all of the SNP markers shown in Table 3 (each SNP marker allele can optionally be present in a heterozygous or homozygous state), each having the resistance genotype as shown in Tables 3 to 5.

In embodiments, the introgressed sequence further comprises the Peronospora resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45; and/or
j) an A genotype in the heterozygous or homozygous state for SNP marker 10 in SEQ ID NO: 50.

In embodiments, the plant comprises the Peronospora resistance-associated genotype at two or more, three or more, four or more, five or more, six or more, or seven or more of the SNP markers of c) to j) above in any combination, optionally at all of the SNP markers of c) to j), each marker allele optionally being present in a heterozygous or homozygous state. In further representative embodiments, the plant comprises the Peronospora resistance-associated genotype at two or more, three or more, four or more, five or more, six or more, seven or more, eight or more, or nine or more of the SNP markers of a) to j) above, optionally at all of the SNP markers of a) to j) in any combination, each marker allele optionally being present in a heterozygous or homozygous state.

The genotype at the SNP markers disclosed herein can be determined by any suitable means known in the art. In representative embodiments, as exemplified in Tables 4 and 5:
the A genotype for SNP marker 1 can be identified in a PCR by amplification of a nucleic acid fragment with a pair of oligonucleotide primers: forward primer of SEQ ID NO: 1 and reverse primer of SEQ ID NO: 4, and probe of SEQ ID NO: 2;
the A genotype for SNP marker 2 can be identified in a PCR by amplification of a nucleic acid fragment with a pair of oligonucleotide primers: forward primer of SEQ ID NO: 6 and reverse primer of SEQ ID NO: 9, and probe of SEQ ID NO: 7;
the A genotype for SNP marker 3 can be identified in a PCR by amplification of a nucleic acid fragment with a pair of oligonucleotide primers: forward primer of SEQ ID NO: 11 and reverse primer of SEQ ID NO: 14, and probe of SEQ ID NO: 12;
the G genotype for SNP marker 4 can be identified in a PCR by amplification of a nucleic acid fragment with a pair of oligonucleotide primers: forward primer of SEQ ID NO: 16 and reverse primer of SEQ ID NO: 19, and probe of SEQ ID NO: 17;
the C genotype for SNP marker 5 can be identified in a PCR by amplification of a nucleic acid fragment with a pair of oligonucleotide primers: forward primer of SEQ ID NO: 21 and reverse primer of SEQ ID NO: 24, and probe of SEQ ID NO: 22;
the A genotype for SNP marker 6 can be identified in a PCR by amplification of a nucleic acid fragment with a pair of oligonucleotide primers: forward primer of SEQ ID NO: 26 and reverse primer of SEQ ID NO: 29, and probe of SEQ ID NO: 27;
the T genotype for SNP marker 7 can be identified in a PCR by amplification of a nucleic acid fragment with a pair of oligonucleotide primers: forward primer of SEQ ID NO: 31 and reverse primer of SEQ ID NO: 34, and probe of SEQ ID NO: 32;
the A genotype for SNP marker 8 can be identified in a PCR by amplification of a nucleic acid fragment with a pair of oligonucleotide primers: forward primer of SEQ ID NO: 36 and reverse primer of SEQ ID NO: 39, and probe of SEQ ID NO: 37;
the G genotype for SNP marker 9 can be identified in a PCR by amplification of a nucleic acid fragment with a pair of oligonucleotide primers: forward primer of SEQ ID NO: 41 and reverse primer of SEQ ID NO: 44, and probe of SEQ ID NO: 42;
the A genotype for SNP marker 10 can be identified in a PCR by amplification of a nucleic acid fragment with a pair of oligonucleotide primers: forward primer of SEQ ID NO: 46 and reverse primer of SEQ ID NO: 49, and probe of SEQ ID NO: 47; and
the G genotype for SNP marker 11 can be identified in a PCR by amplification of a nucleic acid fragment with a pair of oligonucleotide primers: forward primer of SEQ ID NO: 51 and reverse primer of SEQ ID NO: 54, and probe of SEQ ID NO: 52.

In representative embodiments, the plant of the invention comprises the Peronospora resistance-associated genotype at least at the following combination of SNP markers (each individual marker optionally being present in heterozygous or homozygous form):
i. 11 and 1;
ii. 11 and 2;
iii. 11 and 3;
iv. 11 and 4;
v. 11 and 5;
vi. 11 and 6;
vii. 11 and 7;
viii. 11 and 8;
ix. 11, 10, 9 and 8;
x. 11, 10, 9, 8 and 7;
xi. 11, 10, 9, 8, 7 and 6;
xii. 11, 10, 9, 8, 7, 6 and 5;
xiii. 11, 10, 9, 8, 7, 6, 5 and 4;
xiv. 11, 10, 9, 8, 7, 6, 5, 4 and 3;
xv. 11, 10, 9, 8, 7, 6, 5, 4, 3 and 2;
xvi. 11, 10, 9, 8, 7, 6, 5, 4, 3 and 1; or
xvii. 11, 10, 9, 8, 7, 6, 5, 4, 3, 2 and 1.

In representative embodiments, the introgressed sequence comprises one or more, two or more, three or more, four or more, five or more, six or more, seven or more, eight or more, nine or more, ten or more, or all eleven of SEQ ID NO: 5, SEQ ID NO: 10, SEQ ID NO: 15, SEQ ID NO: 20, SEQ ID NO: 25, SEQ ID NO: 30, SEQ ID NO: 35, SEQ ID NO: 40, SEQ ID NO: 45, SEQ ID NO: 50, and/or SEQ ID NO: 55, or a sequence that is at least 80%, 85%, 90%, 95%, 96%, 97%, 98%, or 99% identical to one or more of the foregoing sequences and comprises the indicated resistance-associated SNP marker genotype. Each of the foregoing sequences can optionally be present in a heterozygous or homozygous form.

In a further embodiment, the invention provides a plant according to any of the embodiments described herein wherein said plant comprises at least one copy (e.g., is heterozygous or homozygous) of said Peronospora resistance-conferring introgressed sequence. In embodiments, the plant is heterozygous for the resistance associated genotype at least at one of SNP marker 11 and/or said one or more SNP markers of a) to j) above (when the marker is present in the plant).

In a further embodiment, the invention provides a plant according to any embodiment of the invention wherein said plant comprises two copies (e.g., said plant is homozygous) of said Peronospora resistance-conferring introgressed sequence. In embodiments, the plant is homozygous for the resistance associated genotype at SNP marker 11 and for all of said one or more SNP markers of a) to j) (when the marker is present in the plant).

In a further embodiment, the plant of the invention is an inbred, a dihaploid or a hybrid plant.

In embodiments, the invention provides a plant of deposited Spinacia oleracea line 21BNL002487 or S. oleracea line 21BNL002472, and an F₁ progeny plant thereof.

In a further embodiment, the invention provides a seed that produces a plant (e.g., an S. oleracea plant) according to the invention.

In a further embodiment, the invention provides a seed produced by a plant (e.g., an S. oleracea plant) according to the invention.

In a further embodiment, the spinach plant (e.g., an S. oleracea plant) of the invention is a spinach plant according to any embodiment described herein, wherein S. oleracea line 21BNL002487 or S. oleracea line 21BNL002472, or a progeny or an ancestor thereof, is the source of said Peronospora resistance-conferring introgressed sequence.

In a further embodiment, the spinach plant (e.g., an S. oleracea plant) of the invention is a spinach plant according to any of preceding embodiments, wherein said plant is obtained by crossing S. oleracea line 21BNL002487 or S. oleracea line 21BNL002472, or a progeny or an ancestor thereof, with a spinach plant (e.g., a S. oleracea plant) that does not contain said Peronospora resistance-conferring introgressed sequence.

The invention further encompasses a plant part, cell, organ or tissue obtainable from a spinach plant (e.g., an S. oleracea plant) according to any of preceding embodiments, including but not limited to leaves, stems, roots, flowers or flower parts, shoots, gametophytes, sporophytes, pollen, anthers, microspores, egg cells, zygotes, embryos, meristematic regions, callus tissue, seeds, cuttings, cell or tissue cultures or any other part, cell, organ or tissue of the plant. In embodiments, the plant part, cell, organ or tissue comprises the introgressed sequence that confers Peronospora resistance. In embodiments, the plant part, cell, organ or tissue exhibits the Peronospora resistance according to the invention, particularly when grown to produce a plant.

The invention further provides the use of a spinach plant (e.g., a S. oleracea plant), plant part or seed according to any embodiment of the invention for producing, and optionally harvesting, spinach leaves.

In another aspect, the invention relates to the use of a spinach plant (e.g., a S. oleracea plant), plant part or seed according to any embodiment of the invention, wherein the spinach plant, plant part or seed is a S. oleracea line 21BNL002487 or S. oleracea line 21BNL002472 plant, plant part or seed, or a progeny or an ancestor thereof.

In a further aspect, the invention relates to the use of a spinach plant (e.g., a S. oleracea plant), plant part or seed according to any of the embodiments of the invention to sow a field, a greenhouse, or a plastic house.

In embodiments, the plant according to the invention is male sterile.

In embodiments, the plant according to the invention grows mature spinach leaves (e.g., S. oleracea leaves).

The invention further provides spinach leaves produced by a spinach plant (e.g., a S. oleracea plant) according to any of the embodiments of the invention.

The present invention also relates to a spinach seed, particularly a cultivated spinach seed (e.g., a S. oleracea seed) that grows into a spinach plant according to any of the embodiments of the invention.

The invention additionally relates to the use of a spinach plant according to any of the embodiments to introduce (e.g., introgress) a Peronospora resistance trait of the invention into another spinach plant (e.g., a S. oleracea plant) lacking said Peronospora resistance trait.

### Genetic Sequences, Markers.

The present invention is further directed to a genetic sequence directing or controlling expression of the Peronospora resistance trait in the spinach plant. In a further embodiment, the genetic sequence of the present invention is located on chromosome 3. In a further embodiment of the present invention, the genetic sequence is obtainable from a donor plant that has the genetic background of S. oleracea line 21BNL002487 or S. oleracea line 21BNL002472, or a progeny or an ancestor thereof, and comprises said genetic sequence.

In a further embodiment, the genetic sequence of the present invention is genetically or physically linked to one or more marker loci, which co-segregate with the Peronospora resistance trait (see, e.g., the marker loci of Table 3 and the resistance associated genotypes shown in Tables 3 and 5).

In another embodiment, said genetic sequences of the present invention are characterized by the presence of one or more SNP markers as described in Table 3, having the indicated resistance genotype/allele indicated in Tables 3 to 5 at, for example, one or more, two or more, three or more, four or more, five or more, six or more, seven or more, eight or more, nine or more, or ten or more of the SNP markers in Table 3, in any combination, or all of the SNP markers in Table 3.

In embodiments, the genetic sequence of the present invention comprises a G genotype in the heterozygous or homozygous state for SNP marker 11 in SEQ ID NO: 55.

In embodiments, the genetic sequence further comprises the Peronospora resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45; and/or
j) an A genotype in the heterozygous or homozygous state for SNP marker 10 in SEQ ID NO: 50.

In embodiments, the genetic sequence comprises the Peronospora resistance-associated genotype at two or more, three or more, four or more, five or more, six or more, or seven or more of the resistance-associated genotypes at the SNP markers of c) to j) above in any combination, optionally at all of the SNP markers of c) to j), each marker optionally being present in a heterozygous or homozygous state.

In further representative embodiments, the genetic sequence comprises the Peronospora resistance-associated genotype at two or more, three or more, four or more, five or more, six or more, seven or more, eight or more, or nine or more of the resistance-associated genotypes at the SNP markers of a) to j) above in any combination, optionally at all of the SNP markers of a) to j), each marker optionally being present in a heterozygous or homozygous state.

In representative embodiments, the genetic sequence of the invention comprises the resistance-associated genotypes at least at the following combination of SNP markers:
i. 11 and 1;
ii. 11 and 2;
iii. 11 and 3;
iv. 11 and 4;
v. 11 and 5;
vi. 11 and 6;
vii. 11 and 7;
viii. 11 and 8;
ix. 11, 10, 9 and 8;
x. 11, 10, 9, 8 and 7;
xi. 11, 10, 9, 8, 7 and 6;
xii. 11, 10, 9, 8, 7, 6 and 5;
xiii. 11, 10, 9, 8, 7, 6, 5 and 4;
xiv 11, 10, 9, 8, 7, 6, 5, 4 and 3;
xv. 11, 10, 9, 8, 7, 6, 5, 4, 3 and 2;
xvi. 11, 10, 9, 8, 7, 6, 5, 4, 3 and 1; or
xvii. 11, 10, 9, 8, 7, 6, 5, 4, 3, 2 and 1.

In embodiments, the genetic sequence comprises one or more, two or more, three or more, four or more, five or more, six or more, seven or more, eight or more, nine or more, ten or more, or all eleven of SEQ ID NO: 5, SEQ ID NO: 10, SEQ ID NO: 15, SEQ ID NO: 20, SEQ ID NO: 25, SEQ ID NO: 30, SEQ ID NO: 35, SEQ ID NO: 40, SEQ ID NO: 45, SEQ ID NO: 50, and/or SEQ ID NO: 55, or a sequence that is at least 80%, 85%, 90%, 95%, 96%, 97%, 98%, or 99% identical to one or more of the foregoing sequences and comprises the indicated resistance-associated SNP marker genotype.

The indicated genotypes at the SNP markers of the invention can be determined by any suitable means known in the art. In illustrative but nonlimiting embodiments, as exemplified in Tables 4 and 5:
the A genotype for SNP marker 1 can be identified in a PCR by amplification of a nucleic acid fragment with a pair of oligonucleotide primers: forward primer of SEQ ID NO: 1 and reverse primer of SEQ ID NO: 4, and probe of SEQ ID NO: 2;
the A genotype for SNP marker 2 can be identified in a PCR by amplification of a nucleic acid fragment with a pair of oligonucleotide primers: forward primer of SEQ ID NO: 6 and reverse primer of SEQ ID NO: 9, and probe of SEQ ID NO: 7;
the A genotype for SNP marker 3 can be identified in a PCR by amplification of a nucleic acid fragment with a pair of oligonucleotide primers: forward primer of SEQ ID NO: 11 and reverse primer of SEQ ID NO: 14, and probe of SEQ ID NO: 12;
the G genotype for SNP marker 4 can be identified in a PCR by amplification of a nucleic acid fragment with a pair of oligonucleotide primers: forward primer of SEQ ID NO: 16 and reverse primer of SEQ ID NO: 19, and probe of SEQ ID NO: 17;
the C genotype for SNP marker 5 can be identified in a PCR by amplification of a nucleic acid fragment with a pair of oligonucleotide primers: forward primer of SEQ ID NO: 21 and reverse primer of SEQ ID NO: 24, and probe of SEQ ID NO: 22;
the A genotype for SNP marker 6 can be identified in a PCR by amplification of a nucleic acid fragment with a pair of oligonucleotide primers: forward primer of SEQ ID NO: 26 and reverse primer of SEQ ID NO: 29, and probe of SEQ ID NO: 27;
the T genotype for SNP marker 7 can be identified in a PCR by amplification of a nucleic acid fragment with a pair of oligonucleotide primers: forward primer of SEQ ID NO: 31 and reverse primer of SEQ ID NO: 34, and probe of SEQ ID NO: 32;
the A genotype for SNP marker 8 can be identified in a PCR by amplification of a nucleic acid fragment with a pair of oligonucleotide primers: forward primer of SEQ ID NO: 36 and reverse primer of SEQ ID NO: 39, and probe of SEQ ID NO: 37;
the G genotype for SNP marker 9 can be identified in a PCR by amplification of a nucleic acid fragment with a pair of oligonucleotide primers: forward primer of SEQ ID NO: 41 and reverse primer of SEQ ID NO: 44, and probe of SEQ ID NO: 42;
the A genotype for SNP marker 10 can be identified in a PCR by amplification of a nucleic acid fragment with a pair of oligonucleotide primers: forward primer of SEQ ID NO: 46 and reverse primer of SEQ ID NO: 49, and probe of SEQ ID NO: 47; and
the G genotype for SNP marker 11 can be identified in a PCR by amplification of a nucleic acid fragment with a pair of oligonucleotide primers: forward primer of SEQ ID NO: 51 and reverse primer of SEQ ID NO: 54, and probe of SEQ ID NO: 52.

In a further embodiment, the present invention discloses a kit for the detection (e.g., by genotyping) of the Peronospora resistance trait locus of the invention in a spinach plant, optionally a cultivated S. oleracea plant, wherein said kit comprises at least one PCR oligonucleotide primer pair and probe, selected from:
a forward primer of SEQ ID NO: 1 and reverse primer of SEQ ID NO: 4, and probe of SEQ ID NO: 2;
a forward primer of SEQ ID NO: 6 and reverse primer of SEQ ID NO: 9, and probe of SEQ ID NO: 7;
a forward primer of SEQ ID NO: 11 and reverse primer of SEQ ID NO: 14, and probe of SEQ ID NO: 12;
a forward primer of SEQ ID NO: 16 and reverse primer of SEQ ID NO: 19, and probe of SEQ ID NO: 17;
a forward primer of SEQ ID NO: 21 and reverse primer of SEQ ID NO: 24, and probe of SEQ ID NO: 22;
a forward primer of SEQ ID NO: 26 and reverse primer of SEQ ID NO: 29, and probe of SEQ ID NO: 27;
a forward primer of SEQ ID NO: 31 and reverse primer of SEQ ID NO: 34, and probe of SEQ ID NO: 32;
a forward primer of SEQ ID NO: 36 and reverse primer of SEQ ID NO: 39, and probe of SEQ ID NO: 37;
a forward primer of SEQ ID NO: 41 and reverse primer of SEQ ID NO: 44, and probe of SEQ ID NO: 42;
a forward primer of SEQ ID NO: 46 and reverse primer of SEQ ID NO: 49, and probe of SEQ ID NO: 47; and
a forward primer of SEQ ID NO: 51 and reverse primer of SEQ ID NO: 54, and probe of SEQ ID NO: 52.

The present invention also discloses the use of the SNP markers according to the invention for diagnostic selection and/or genotyping of the Peronospora resistance trait locus in a spinach plant, particularly a cultivated spinach plant (e.g., a S. oleracea plant)

The present invention further discloses the use of some or all of these SNP markers for identifying in a spinach plant, particularly a cultivated spinach plant, more particularly a S. oleracea plant according to the invention, the presence of a Peronospora resistance trait locus and/or for monitoring the introgression of the Peronospora resistance trait locus in a spinach plant, particularly a cultivated spinach plant, particularly a S. oleracea plant according to the invention and as described herein.

The invention further discloses a polynucleotide (amplification product) obtainable in a PCR reaction involving the use of at least one oligonucleotide primer disclosed herein: SEQ ID NO: 1, SEQ ID NO: 4, SEQ ID NO: 6, SEQ ID NO: 9, SEQ ID NO: 11, SEQ ID NO: 14, SEQ ID NO: 16, SEQ ID NO: 19, SEQ ID NO: 21, SEQ ID NO: 24, SEQ ID NO: 26, SEQ ID NO: 29, SEQ ID NO: 31, SEQ ID NO: 34, SEQ ID NO: 36, SEQ ID NO: 39, SEQ ID NO: 41, SEQ ID NO: 44, SEQ ID NO: 46, SEQ ID NO: 49, SEQ ID NO: 51, and/or SEQ ID NO: 54, and reacting with probes of SEQ ID NO: 2, SEQ ID NO: 7, SEQ ID NO: 12, SEQ ID NO: 17, SEQ ID NO: 22, SEQ ID NO: 27, SEQ ID NO: 32, SEQ ID NO: 37, SEQ ID NO: 42, SEQ ID NO: 47 and/or SEQ ID NO: 52, respectively (see Tables 4 and 5). The invention also provides a polynucleotide (amplification product) obtainable in a PCR reaction using a pair of PCR oligonucleotide primers selected from: SEQ ID NO: 1 and SEQ ID NO: 4; SEQ ID NO: 6 and SEQ ID NO: 9; SEQ ID NO: 11 and SEQ ID NO: 14; SEQ ID NO: 16 and SEQ ID NO: 19; SEQ ID NO: 21 and SEQ ID NO: 24; SEQ ID NO: 26 and SEQ ID NO: 29; SEQ ID NO: 31 and SEQ ID NO: 34; SEQ ID NO: 36 and SEQ ID NO: 39; SEQ ID NO: 41 and SEQ ID NO: 44; SEQ ID NO: 46 and SEQ ID NO: 49; and/or SEQ ID NO: 51 and SEQ ID NO: 54, and reacting with probes of SEQ ID NO: 2, SEQ ID NO: 7, SEQ ID NO: 12, SEQ ID NO: 17, SEQ ID NO: 22, SEQ ID NO: 27, SEQ ID NO: 32, SEQ ID NO: 37, SEQ ID NO: 42, SEQ ID NO: 47 and/or SEQ ID NO: 52, respectively.

Also contemplated herein is a polynucleotide that has at least 80%, particularly at least 85%, particularly at least 90%, particularly at least 95%, particularly at least 96%, particularly at least 97%, particularly at least 98%, particularly at least 99% nucleotide sequence identity with the nucleotide sequence of said amplification product and/or a polynucleotide comprising a nucleotide sequence that hybridizes to the nucleotide sequence of said amplification product obtainable in the above PCR reaction.

In embodiments, the amplification product corresponds to an amplification product obtainable from S. oleracea line 21BNL002487 or S. oleracea line 21BNL002472, or a progeny or an ancestor thereof, when using the same primer/primer pair, wherein said amplification product comprises the Peronospora resistance-conferring introgressed sequence of the invention, or is at least 80%, 85%, 90%, 95%, 96%, 97%, 98% or 99% identical thereto.

The amplification product according to the invention and described herein can be used for generating or developing new primers and/or probes that can be used for identifying the Peronospora resistance trait locus of the invention. The present invention therefore further relates in one embodiment to derived markers, particularly to derived primers or probes, developed from an amplification product according to the invention and as described herein and by methods known in the art, which derived markers are genetically linked to the Peronospora resistance trait locus of the invention.

### Methods of Breeding.

The invention also provides methods for producing a spinach plant having enhanced resistance to Peronospora effusa (e.g., as compared with a control). In embodiments, the method comprises:
crossing a first spinach plant (e.g., a S. oleracea plant, optionally a cultivated S. oleracea plant) according to the invention (e.g., a “donor” plant”) with a second spinach plant that lacks the introgressed sequence of the invention conferring resistance to Peronospora effusa (e.g., a “recipient” plant) to produce progeny plants; and
selecting a progeny plant comprising said introgressed sequence conferring resistance to Peronospora effusa, thereby producing a plant with enhanced resistance to Peronospora effusa.

Optionally, the second spinach plant is susceptible to one or more of Peronospora effusa races Pe: 1-18. In embodiments, the first spinach plant is a cultivated spinach plant. In embodiments, the first spinach plant is a S. oleracea plant. In embodiments, the second spinach plant is a cultivated spinach plant. In embodiments, the second spinach plant is a S. oleracea plant.

According to representative embodiments, the method can optionally further comprise:
selfing the selected progeny plant or crossing the selected progeny with another spinach plant (e.g., a S. oleracea plant) to produce further progeny. In embodiments, further progeny are selected and selfed/crossed (including backcrossed) for 2 to 10 more generations, optionally to create an inbred line comprising the introgressed sequence conferring Peronospora resistance according to the invention.

In a further embodiment, the invention relates to the method of any one of the embodiments described herein wherein the first spinach plant of step a) is S. oleracea line 21BNL002487 or S. oleracea line 21BNL002472, or a progeny or an ancestor thereof comprising the introgressed sequence of the invention. Optionally, according to this embodiment the second spinach plant lacks an introgressed sequence of the invention conferring resistance to Peronospora effusa.

In exemplary embodiments, said selecting step comprises detecting (e.g., genotyping) the presence of a resistance-associated genotype at one or more SNP markers in the chromosome interval defined by SNP marker 11 to 8, 7, 6, 5, 4 or 3 (inclusive). In embodiments, the method comprises detecting the presence of a resistance-associated genotype at one or more markers in the chromosome interval defined by SNP marker 11 to 2 or 1 (inclusive). In embodiments, the method comprises detecting the presence of a resistance-associated genotype at one or more markers in the chromosome interval is defined by SNP marker 11 to 3 (inclusive). The method can comprise detecting a resistance-associated genotype at any combination of the markers in the chromosome interval, for example, the non-limiting list of SNP markers as shown in Tables 3 to 5.

In embodiments, said selecting step comprises detecting (e.g., by genotyping) the presence of the resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45;
j) an A genotype in the heterozygous or homozygous state for SNP marker 10 in SEQ ID NO: 50 and/or
k) a G genotype in the heterozygous or homozygous state for SNP marker 11 in SEQ ID NO: 55.

In embodiments, the selecting step comprises detecting (e.g., by genotyping) the presence of the Peronospora resistance-associated genotype at two or more, three or more, four or more, five or more, six or more, seven or more, or eight or more of the SNP markers of c) to k) above in any combination, optionally at all of the SNP markers of c) to k), each marker optionally being present in a heterozygous or homozygous state.

In further representative embodiments, the selecting step comprises detecting (e.g., by genotyping) the presence of the Peronospora resistance-associated genotype at two or more, three or more, four or more, five or more, six or more, seven or more, eight or more, nine or more, or ten or more of the SNP markers of a) to k) above in any combination, optionally at all of the SNP markers of a) to k), each marker optionally being present in a heterozygous or homozygous state.

In embodiments, the method comprises detecting (e.g., by genotyping) the presence of the Peronospora resistance-associated genotype at SNP marker 11 and one or more of 8, 7, 6, 5, 4 and/or 3. In embodiments, the method comprises detecting (e.g., by genotyping) the presence of the Peronospora resistance-associated genotype at SNP markers 11 and one or both of 2 and 1. In embodiments, the method comprises detecting (e.g., by genotyping) the presence of the Peronospora resistance-associated genotype at SNP markers 11 and 3. The method can further comprise detecting the presence of the Peronospora resistance-associated genotype at any combination of the markers in the chromosome interval, for example, the non-limiting list of SNP markers as shown in Tables 3 to 5, having the indicated resistance genotype (allele).

In embodiments of the methods according to the invention, the method comprises detecting (e.g., by genotyping) the presence of the resistance associated genotype (e.g., as described in Tables 3 to 5) at least at the following combination of SNP markers (each individual marker optionally being present in heterozygous or homozygous form):
i. 11 and 1;
ii. 11 and 2;
iii. 11 and 3;
iv. 11 and 4;
v. 11 and 5;
vi. 11 and 6;
vii. 11 and 7;
viii. 11 and 8;
ix. 11, 10, 9 and 8;
x. 11, 10, 9, 8 and 7;
xi. 11, 10, 9, 8, 7 and 6;
xii. 11, 10, 9, 8, 7, 6 and 5;
xiii. 11, 10, 9, 8, 7, 6, 5 and 4;
xiv. 11, 10, 9, 8, 7, 6, 5, 4 and 3;
xv. 11, 10, 9, 8, 7, 6, 5, 4, 3 and 2;
xvi. 11, 10, 9, 8, 7, 6, 5, 4, 3 and 1; or
xvii. 11, 10, 9, 8, 7, 6, 5, 4, 3, 2 and 1.

The invention also provides methods for producing a F1 hybrid spinach plant (e.g., a S. oleracea plant) with enhanced resistance to Peronospora effusa (e.g., as compared with a control plant). In embodiments, the method comprises crossing an inbred (including dihaploid) spinach plant of the invention with a different inbred spinach plant to produce a F1 hybrid progeny. According to this method, the second spinach plant may or may not comprise the introgressed sequence conferring the Peronospora resistance of the invention. In embodiments where the second spinach plant does not comprise the introgressed sequence of the invention, the different inbred spinach plant is optionally susceptible to one or more of Peronospora effusa races Pe: 1-18.

In embodiments, the first spinach plant is a cultivated spinach plant. In embodiments, the first spinach plant is a S. oleracea plant. In embodiments, the second spinach plant is a cultivated spinach plant. In embodiments, the second spinach plant is a S. oleracea plant.

In embodiments, the method of producing a F1 hybrid spinach plant with enhanced Peronospora resistance further comprises detecting (e.g., by genotyping) the presence of a resistance-associated genotype at one or more SNP markers in the chromosome interval defined by SNP marker 11 to 8, 7, 6, 5, 4 or 3 (inclusive). In embodiments, the method comprises detecting the presence of a resistance-associated genotype at one or more markers in the chromosome interval defined by SNP marker 11 to 2 or 1 (inclusive). In embodiments, the method comprises detecting the presence of a resistance-associated genotype at one or more markers in the chromosome interval is defined by SNP marker 11 to 3 (inclusive). The method can comprise detecting a resistance-associated genotype at any combination of the markers in the chromosome interval, for example, the non-limiting list of SNP markers as shown in Tables 3, 4 and 5.

Optionally, the method of producing a F1 hybrid spinach plant with enhanced Peronospora resistance further comprises detecting (e.g., by genotyping) the presence of a resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45;
j) an A genotype in the heterozygous or homozygous state for SNP marker 10 in SEQ ID NO: 50; and/or
k) a G genotype in the heterozygous or homozygous state for SNP marker 11 in SEQ ID NO: 55.

In any of the breeding methods of the invention, the method optionally further comprises testing the plant, progeny or F1 hybrid plant for resistance against Peronospora, for example, one or more of Peronospora effusa races Pe: 1-18. In embodiments, the plant, progeny or F1 hybrid has resistance against at least Peronospora effusa races Pe: 1-18.

According to any of the breeding methods of the invention that comprise detecting (e.g., genotyping) a resistance-associated genotype at one or more SNP markers, the method can comprise providing or obtaining a nucleic acid sample (e.g., a DNA sample, optionally a genomic DNA sample) from the plant and detecting the presence of the resistance-associated genotype(s) in the nucleic acid sample.

### Methods of Selection.

The invention also contemplates methods of identifying spinach plants having enhanced resistance to Peronospora effusa and having at least one copy of an introgressed sequence of the invention (e.g., the plant is heterozygous or homozygous). In embodiments, the method comprises detecting (e.g., by genotyping) the presence of a resistance-associated genotype at one or more SNP markers in the chromosome interval defined by SNP marker 11 to 8, 7, 6, 5, 4 or 3 (inclusive). In embodiments, the method comprises detecting the presence of a resistance-associated genotype at one or more markers in the chromosome interval defined by SNP marker 11 to 2 or 1 (inclusive). In embodiments, the method comprises detecting the presence of a resistance-associated genotype at one or more markers in the chromosome interval is defined by SNP marker 11 to 3 (inclusive). The method can comprise detecting a resistance-associated genotype at any combination of the markers in the chromosome interval, for example, the non-limiting list of SNP markers as shown in Tables 3, 4 and 5.

In exemplary embodiments, the method comprises the step of detecting the presence of a resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45;
j) an A genotype in the heterozygous or homozygous state for SNP marker 10 in SEQ ID NO: 50; and/or
k) a G genotype in the heterozygous or homozygous state for SNP marker 11 in SEQ ID NO: 55,
thereby identifying a spinach plant with enhanced resistance to Peronospora effusa.

In embodiments, the plant is a cultivated plant (e.g., a cultivated S. oleracea).

In embodiments, the method further comprises selecting a spinach plant comprising said resistance-associated genotype at the one or more SNP markers, and crossing the selected spinach plant with a second spinach plant to produce a progeny spinach plant that comprises the resistance-associated genotype at the one or more SNP markers and is resistant to Peronospora (e.g., comprises the introgressed sequence of the invention conferring Peronospora resistance in the heterozygous or homozygous state). In embodiments, the second spinach plant is different from the selected spinach plant. In embodiments, the second spinach plant is a S. oleracea. In embodiments, the second spinach plant does not comprise the introgressed sequence conferring Peronospora resistance of the invention, and optionally is susceptible to one or more of Peronospora effusa races Pe: 1-18. In embodiments, the second spinach plant does comprise said introgressed sequence.

In another embodiment, the invention relates to a method of identifying a spinach plant (e.g., a S. oleracea plant, optionally a cultivated S. oleracea plant) comprising the Peronospora resistance-conferring introgressed sequence of the invention, wherein said method comprises the steps of:
providing a population of spinach plants segregating for the Peronospora resistance trait;
screening the segregating population for a member exhibiting resistance to Peronospora, wherein said trait can be identified by the presence of a Peronospora resistance-conferring introgressed sequence of the invention.

Optionally, the step of determining the presence of the introgressed sequence comprises detecting (e.g., by genotyping) the presence of a resistance-associated genotype at one or more SNP markers in the chromosome interval defined by SNP marker 11 to 8, 7, 6, 5, 4 or 3 (inclusive). In embodiments, the method comprises detecting the presence of a resistance-associated genotype at one or more markers in the chromosome interval defined by SNP marker 11 to 2 or 1 (inclusive). In embodiments, the method comprises detecting the presence of a resistance-associated genotype at one or more markers in the chromosome interval is defined by SNP marker 11 to 3 (inclusive). The method can comprise detecting a resistance-associated genotype at any combination of the markers in the chromosome interval, for example, the non-exhaustive list of SNP markers as shown in Tables 3, 4 and 5.

In embodiments, determining the presence of the introgressed sequence comprises detecting (e.g., genotyping) the presence of a resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45;
j) an A genotype in the heterozygous or homozygous state for SNP marker 10 in SEQ ID NO: 50; and/or
k) a G genotype in the heterozygous or homozygous state for SNP marker 11 in SEQ ID NO: 55; and
selecting a member of the segregating population comprising the Peronospora resistance-conferring introgressed sequence of the invention.

In any of the selection methods of the invention that comprise detecting (e.g., genotyping) a resistance-associated genotype at one or more SNP markers, the method can comprise providing or obtaining a nucleic acid sample (e.g., a DNA sample, optionally a genomic DNA sample) from the plant and detecting the presence of the resistance-associated genotype(s) in the nucleic acid sample.

As another aspect, the invention further provides methods for assessing the genotype of a spinach plant (e.g., a S. oleracea plant, optionally a cultivated S. oleracea plant), exhibiting resistance to Peronospora effusa, said method comprising the steps of:
providing a nucleic acid sample (e.g., DNA, optionally genomic DNA) from said plant, and,
detecting in said nucleic acid sample the presence or absence of a resistance-associated genotype at one or more SNP markers in the chromosome interval defined by SNP marker 11 to 8, 7, 6, 5, 4 or 3 (inclusive). In embodiments, the method comprises detecting the presence or absence of a resistance-associated genotype at one or more markers in the chromosome interval defined by SNP marker 11 to 2 or 1 (inclusive). In embodiments, the method comprises detecting the presence or absence of a resistance-associated genotype at one or more markers in the chromosome interval is defined by SNP marker 11 to 3 (inclusive). The method can comprise detecting the presence or absence of a resistance-associated genotype at any combination of the markers in the chromosome interval, for example, the non-exhaustive list of SNP markers as shown in Tables 3, 4 and 5;
thereby assessing the genotype of a spinach plant.

In embodiments, the method comprises detecting the presence or absence of a resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45;
j) an A genotype in the heterozygous or homozygous state for SNP marker 10 in SEQ ID NO: 50; and/or
k) a G genotype in the heterozygous or homozygous state for SNP marker 11 in SEQ ID NO: 55,

In embodiments, the methods of the invention for identifying, selecting or genotyping plants further comprise detecting (e.g., by genotyping) the presence of the Peronospora resistance-associated genotype at two or more, three or more, four or more, five or more, six or more, seven or more, or eight or more of the SNP markers of c) to k) above in any combination, optionally at all of the SNP markers of c) to k), each marker optionally being present in a heterozygous or homozygous state.

In further representative embodiments, the methods further comprises detecting (e.g., by genotyping) the presence of the Peronospora resistance-associated genotype at two or more, three or more, four or more, five or more, six or more, seven or more, eight or more, nine or more, or ten or more of the SNP markers of a) to k) above in any combination, optionally at all of the SNP markers of a) to k), each marker optionally being present in a heterozygous or homozygous state.

In embodiments of the foregoing methods of identifying, selecting or genotyping plants, the method comprises detecting (e.g., by genotyping) the presence of the resistance associated genotype (e.g., as described in Tables 3 to 5) at least one of the following combinations of SNP markers (each individual marker optionally being present in heterozygous or homozygous form):
i. 11 and 1;
ii. 11 and 2;
iii. 11 and 3;
iv. 11 and 4;
v. 11 and 5;
vi. 11 and 6;
vii. 11 and 7;
viii. 11 and 8;
ix. 11, 10, 9 and 8;
x. 11, 10, 9, 8 and 7;
xi. 11, 10, 9, 8, 7 and 6;
xii. 11, 10, 9, 8, 7, 6 and 5;
xiii. 11, 10, 9, 8, 7, 6, 5 and 4;
xiv. 11, 10, 9, 8, 7, 6, 5, 4 and 3;
xv. 11, 10, 9, 8, 7, 6, 5, 4, 3 and 2;
xvi. 11, 10, 9, 8, 7, 6, 5, 4, 3 and 1; or
xvii. 11, 10, 9, 8, 7, 6, 5, 4, 3, 2 and 1.

In embodiments, the methods of the invention comprise determining whether the plant is heterozygous or homozygous for the introgressed sequence of the invention conferring Peronospora resistance.

In representative embodiments, the methods of the invention comprise detecting the presence of one or more of SEQ ID NO: 5, SEQ ID NO: 10, SEQ ID NO: 15, SEQ ID NO: 20, SEQ ID NO: 25, SEQ ID NO: 30, SEQ ID NO: 35, SEQ ID NO: 40, SEQ ID NO: 45, SEQ ID NO: 50, and/or SEQ ID NO: 55, or a sequence that is at least 80%, 85%, 90%, 95%, 96%, 97%, 98%, 99% or more identical to one or more of the foregoing sequences and comprises the indicated resistance-associated SNP marker genotype.

In embodiments, the methods of the invention further comprise testing the identified or genotyped plant for resistance against Peronospora, for example, one or more of Peronospora effusa races Pe: 1-18. In embodiments, the plant has resistance against at least Peronospora effusa races Pe: 1-18.

### Uses.

The present invention also relates to the use of Peronospora resistant-propagating material obtainable from a spinach plant according to any of the embodiments of the invention for growing a spinach plant in order to produce Peronospora resistant spinach plants, seeds, and/or leaves. Peronospora resistance may be assessed in a standard assay, for example, an assay as described in Example 1 below.

In embodiments, the invention provides for a method of producing spinach seed, the method comprising growing a spinach plant from a seed according to any embodiment of the invention, and allowing the plant to produce further spinach seed, and optionally collecting the seed.

The present invention also relates to the use of Peronospora resistant-propagating material obtainable from a spinach plant according to any of the embodiments of the invention for producing spinach leaves.

### Seed Deposit Information

Applicants have made a deposit of at least 625 seeds of Spinacia oleracea line 21 BNL487 with the NCIMB (NCIMB Limited, Ferguson Building, Craibstone Estate, Bucksburn, Aberdeen AB21 9YA, Scotland) on 23 Nov. 2021 under NCIMB Accession No. NCIMB 43893. Applicants have made a deposit of at least 625 seeds of Spinacia oleracea line 21BNL002472 with the NCIMB on 21 Oct. 2022 under NCIMB Accession No. NCIMB 44060.

Applicants elect for the expert solution and request that the deposited material be released only to an Expert according to Rule 32 (1) EPC or corresponding laws and rules of other countries or treaties (Expert Witness clause), until the mention of the grant of the patent publishes, or from 20 years from the date of filing if the application is refused, withdrawn or deemed to be withdrawn.

These deposits of Spinacia oleracea line 21BNL002487 and Spinacia oleracea line 21BNL002472 will be maintained in the NCIMB depository, which is a public depository, for a period of 30 years, or 5 years after the most recent request, or for the effective life of the patent, whichever is longer, and will be replaced if any of the deposited seed becomes nonviable during that period. Additionally, Applicants have satisfied all the requirements of 37 C.F.R. §§ 1.801-1.809, including providing an indication of the viability of the samples. Access to these deposits will be made available during the pendency of this application to the Commissioner upon request. Upon the issuance of a patent on the variety, the variety will be irrevocably and without restriction released to the public by providing access to the deposits of at least 625 seeds of the deposited lines with the NCIMB. Applicants impose no restrictions on the availability of the deposited material from the NCIMB; however, Applicants have no authority to waive any restrictions imposed by law on the transfer of biological material or its transportation in commerce. Applicants do not waive any infringement of its rights granted under this or any other patent or under any plant variety protection rights (e.g., U.S. Plant Variety Protection Act, 7 USC § 2321 et seq.).

The invention will now be described with reference to the following examples. It will be appreciated by those skilled in the art that these examples do not limit the scope of the claims to the invention, but are rather intended to be exemplary of certain embodiments. Other embodiments of the invention may be practiced without departing from the spirit and the scope of the invention, the scope of which is defined by the disclosure and the appended claims.

### EXAMPLES

### Example 1. Disease Test for Peronospora effusa

Peronospora effusa disease tests are done in climate chambers with high humidity. Day length is 16 hours and during the day the temperature is 18° C. and relative humidity (RH) about 85%. During the night the temperature is 15° C. and the RH about 100%. Before inoculation of a test, the spores of the Peronospora effusa pathogen are multiplied on varieties susceptible to the specific isolate. Disease testing for resistance is performed using various Peronospora effusa isolates and races.

Peronospora effusa isolates are characterized and classified on a differential set as defined by IWGP (International Working Group on Peronospora; see Table 1).

TABLE 1
Reactions of Peronospora effusa Pe: 1-19 to the IWGP set of differentials

| | Differentials | | | | | | | | | | |
|---|---|---|---|---|---|---|---|---|---|---|---|
| | | NIL | NIL | NIL | NIL | NIL | NIL | | | | |
| Race | Viroflay | 5 | 3 | 4 | 6 | 1 | 2 | Pigeon | Caladonia | Meerkat | Hydrus |
| Pe: 1 | + | − | − | − | − | − | − | − | − | − | − |
| Pe: 2 | + | − | + | − | + | − | − | − | − | − | − |
| Pe: 3 | + | + | − | − | − | − | − | − | − | − | − |
| Pe: 4 | + | + | + | − | − | − | − | − | − | − | − |
| Pe: 5 | + | + | − | + | − | − | − | − | − | − | − |
| Pe: 6 | + | + | + | + | + | − | − | − | − | − | − |
| Pe: 7 | + | + | + | + | − | − | − | − | − | − | − |
| Pe: 8 | + | + | − | + | + | + | − | − | − | − | − |
| Pe: 9 | + | + | − | + | + | − | − | − | − | − | − |
| Pe: 10 | + | + | + | + | + | + | − | − | − | − | − |
| Pe: 11 | + | + | − | + | − | − | + | − | − | − | − |
| Pe: 12 | + | + | − | + | + | + | + | − | − | − | − |
| Pe: 13 | + | + | + | + | − | (−) | + | − | − | − | − |
| Pe: 14 | + | + | − | + | + | + | + | + | − | − | − |
| Pe: 15 | + | + | + | − | − | − | − | − | + | − | − |
| Pe: 16 | + | + | − | + | − | − | + | + | − | + | − |
| Pe: 17 | + | + | + | + | + | + | + | + | + | (−) | − |
| Pe: 18 | + | + | + | + | − | − | + | + | + | + | − |
| Pe: 19 | + | + | − | + | + | + | + | + | − | + | + |

Legend: + = susceptible,
− = resistant,
(−) intermediate resistant

Before inoculation of test material, the leaves with spores are harvested and the spores rinsed from the leaves with water. The concentration of the spore suspension is adjusted to 100,000 spores per ml. The spore suspension is sprayed over 1-week-old plants (on cotyledons). Seven to 10 days after inoculation the observation/selection can be done. In general, the cotyledons of the susceptible plants are fully covered with spores. Depending on the Peronospora effusa isolate used, the cotyledons of a resistant plant will show no or sparse sporulation.

### Example 2. Strategy to Introduce Peronospora effusa Resistance from Wild Spinach into Cultivated S. oleracea

The inventors determined that a wild Spinacia turkestanica accession KK016 (CGN080741) has a broad spectrum resistance to Peronospora effusa. Peronospora resistance from this wild S. turkestanica source was introduced into a cultivated S. oleracea plant having a fixed Peronospora resistance, followed by backcrossing, and then selfed to fix the resistance in the S. oleracea genetic background. The genetic elements underlying the resistances were tightly linked on chromosome 3, and evaluation of the resistance spectra against a set of Peronospora isolates and molecular markers were used to identify recombination events combining the desired resistance alleles.

The resulting line was crossed with a commercial female S. oleracea parent line. F3 seeds from this pedigree were designated as line 21BNL002487 and were deposited with NCIMB on 23 Nov. 2021 under Deposit No. NCIMB 43893. This F3 line is fixed (i.e., homozygous) for the P. effusa resistance, but is otherwise still segregating at many loci.

The two resistance sources are complementary and combine to provide a broader resistance spectrum to Peronospora effusa as shown in Table 2 below. Both resistances are characterized as dominant (high resistance observed in both heterozygotes and homozygotes), qualitative in nature, and exhibit monogenic segregation.

TABLE 2
Combining resistances to P. effusa.

| | P. effuse race | | | | | | | | | | | | | | | | | |
|---|---|---|---|---|---|---|---|---|---|---|---|---|---|---|---|---|---|---|
| | Pe: | Pe: | Pe: | Pe: | Pe: | Pe: | Pe: | Pe: | Pe: | Pe: | Pe: | Pe: | Pe: | Pe: | Pe: | Pe: | Pe: | Pe: |
| | 1 | 2 | 3 | 4 | 5 | 6 | 7 | 8 | 10 | 11 | 12 | 13 | 14 | 15 | 16 | 17 | 18 | 19 |
| S. oleracea | − | − | − | − | − | − | − | + | + | − | + | − | + | − | − | + | − | + |
| source | | | | | | | | | | | | | | | | | | |
| S. turkestanica | − | − | − | + | − | − | − | − | − | − | − | − | − | − | + | − | − | + |
| source | | | | | | | | | | | | | | | | | | |

Legend: + = susceptible,
− = resistant

Starting with the BC5 (backcross 5) generation, molecular markers were used to identify smaller introgressions on chromosome 3 that retained both desired resistances. The selection process had to be carried out with careful evaluation to retain the full resistance spectra, while minimizing linkage drag and undesirable phenotypes from the wild spinach genetics, and further avoiding dwarfism and other negative agronomic phenotypes that are frequently observed when combining resistances.

Eventually, the resistance was localized to a region of less than 350 KB and designated as “ADQ”. This ADQ resistance maintains the full spectra of qualitative Peronospora resistance from both parental sources (see Table 2 above), conferring protection to Pe: 1-18 in both homozygous and heterozygous plants, while avoiding linkage drag from the wild spinach parent and other adverse phenotypes such as dwarfism.

### Example 3. Mapping and Genotyping the ADQ Peronospora Resistance

### Assay Development

### Step 1: Fingerprinting: Identification of the Introgression

Based on fingerprinting studies, a first estimation of the location of the ADQ resistance was a 119 MB interval on linkage group 3.

### Step 2: ADQ Associated Haplotype SNPs

Variants from Step 1 with different allelic states between the resistant backcross line and recurrent parent across the ADQ introgression were selected and converted into Taqman™ PCR assays to detect SNPs.

Marker 11 is strongly associated with the ADQ Peronospora resistance, and the resistance-associate genotype at marker 11 (see, Tables 3 to 5) is essentially diagnostic, especially when combined with the Peronospora resistance spectrum. In embodiments, to increase the accuracy of predicting the presence of the ADQ Peronospora resistance, for example in the absence of phenotypic data on the Peronospora resistance spectrum and/or across more genetic backgrounds, the presence of the resistance-associated genotype (see, Tables 3 to 5) at additional markers from Table 3 optionally can be assessed, in particular along the chromosome interval defined by marker 11 to 3. Markers 2 and 1 are physically much more distant than the markers within the interval defined by markers 11 to 3 (see, e.g., the physical positions in Table 4), and therefore there would be expected to be more recombination between the chromosome interval defined by markers 11 to 3 and markers 2 and 1.

Accordingly, one or more of the markers in Table 3 can be employed to identify spinach plants comprising the ADQ Peronospora resistance, to follow the resistance for introgression into new lines, and the like. For example, marker 11 can be used alone or in conjunction with one or more of markers 1 to 10.

TABLE 3

| Marker | ADQ | Alternative |
|---|---|---|
| No. | genotype | genotype |
| 1 | A/A | G/G |
| 2 | A/A | G/G |
| 3 | A/A | C/C |
| 4 | G/G | A/A |
| 5 | C/C | A/A |
| 6 | A/A | G/G |
| 7 | T/T | A/A |
| 8 | A/A | G/G |
| 9 | G/G | A/A |
| 10 | A/A | G/G |
| 11 | G/G | A/A |

The physical positions of SNP markers associated with the ADQ Peronospora effusa resistance and PCR primers/probes for detection of these SNPs are shown in Table 4 below. All physical positions are reported with respect to the spinach-Viroflay_v3 reference genome published by Hulse-Kemp et al. (Plant Genome. 2021; 14:e20101).

TABLE 4
Marker 1 at 143976549 bp on Spov3_chr3 (based on reference spinach_Viroflay_v3 )
Target sequence (SEQ ID NO: 5)
AACCTAAAATGTGAAACACTATATGATAAGTTGTTCATTAAAAAACTGCCAATAATCCAATATTTATAATTCCT
TG[A/G]ATTATCATTTTAAGAACGATTTAAAGGATTTTTTTACTAAAATGATGAACAATAATTAAGAAGACAT
TTTGAACTATTTGGTATTGATAGT
>Resistance allele A/A
>Primer forward (underlined) (SEQ ID NO: 1)
GTGAAACACTATATGATAAGTTG
>Probe (double underlined)
Detect the resistant allele: TCGTTCTTAAAATGATAATTCA(SEQ ID NO: 2)
Detect the susceptible allele: CGTTCTTAAAATGATAATCCA (SEQ ID NO: 3)
>Primer reverse (underlined)
CCAAATAGTTCAAAATGTCTTC (SEQ ID NO: 4)
Marker 2 at 143763913 bp on Spov3_chr3 (based on reference spinach_Viroflay_v3)
Target sequence (SEQ ID NO: 10)
GAACATGCATTGCAAACAAATCTCAATATCCATCCTACTAAAGACCCTATTCTACACGTATCGACTACTT[A/G]ACCAT
GCTAAGACATTTGACTTAATCCAGAATCGAATTCTATCTACATCACTGCCTTTGTGGCACTTTCCACAAAAATCAAAAGA
GTAATGGTTGGCAAGGTGCCTACTTTATT
>Resistance allele A/A
>Primer forward (underlined) (SEQ ID NO: 6)
TGCAAACAAATCTCAATATCCATCC
>Probe (double underlined)
Detect the resistant allele: ACGTATCGACTACTTAACC (SEQ ID NO: 7)
Detect the susceptible allele: TATCGACTACTTGACCA (SEQ ID NO: 8)
>Primer reverse (underlined)
GCACCTTGCCAACCATTACTC (SEQ ID NO: 9)
Marker 3 at 148561736 bp on Spov3_chr3 (based on reference spinach_Viroflay_v3)
Target sequence (SEQ ID NO: 15)
TATGGGTTTACGAGACATGCACATAATATGGCAACCCTCTAAGATTCAA[A/C]CCTATCTCAACAATGATATAACTTT
TGTTGAATTGGTTTCATTGGGAACCAAGCGTTTAAACTTTTC
>Resistance allele A/A
>Primer forward (underlined) (SEQ ID NO: 11)
CGAGACATGCACATAATATGGCA
>Probe (double underlined)
Detect the resistant allele: TCTAAGATTCAAACCTATCTC (SEQ ID NO: 12)
Detect the susceptible allele: TCTAAGATTCAACCCTATC (SEQ ID NO: 13)
>Primer reverse (underlined)
AAACGCTTGGTTCCCAATGAAAC (SEQ ID NO: 14)
Marker 4 at 148837818 bp on Spov3_chr3 (based on reference spinach_Viroflay_v3)
Target sequence (SEQ ID NO: 20)
GTATGCTTATACGTTACAAGTCGATGAAAAGAGTGACATCTACAGTTACGGGGTGGTACTATTGGAGATATT[A/G]AGCG
GGAAAAGATCAGTACAACAAGAATTCGGTGAAGGGAACAGNGTN
>Resistance allele G/G
>Primer forward (underlined) (SEQ ID NO: 16)
ACGTTACAAGTCGATGAAAAGAGTG
>Probe (double underlined)
Detect the resistant allele: TACTATTGGAGATATTGAGC (SEQ ID NO: 17)
Detect the susceptible allele: ACTATTGGAGATATTAAGCG (SEQ ID NO: 18)
>Primer reverse (underlined)
CCTTCACCGAATTCTTGTTGTACTG (SEQ ID NO: 19)
Marker 5 at 148982041 bp on Spov3_chr3 (based on reference spinach_Viroflay_v3)
Target sequence (SEQ ID NO: 25)
GTAGTTAATTCTTGTCTTTGAGCAACCTTTCTCTGTGTTATATTGTATGGTGTATGTGATTTTTTT
TTGCTGGATCAC[A/C]ATGGTATACTTGTGTTTTTGTTTTTATAGTTAAATCTGTGTGCTCCT
GCAGGTTCTTTGTGGGAATGGAAAAGCAGCGTTGTTCGAAATTTGGGA
>Resistance allele C/C
>Primer forward (underlined) (SEQ ID NO: 21)
CTTGTCTTTGAGCAACCTTTCTCTG
>Probe (double underlined)
Detect the resistant allele: TTGCTGGATCACCAT (SEQ ID NO: 22)
Detect the susceptible allele: TTGCTGGATCACAATG (SEQ ID NO: 23)
>Primer reverse (underlined)
CGAACAACGCTGCTTTTCCAT (SEQ ID NO: 24)
Marker 6 at 149022257 bp on Spov3_chr3 (based on reference spinach_Viroflay_v3)
Target sequence (SEQ ID NO: 30)
TGCTTTTATTAGCTTACAGGAAGGAGCGTTTTCGCAGTCAATGTGGGTTGTCTC[A/G]TATCGATGACCT
GTTCAAAGTCGGGGAGCTAAGACANCCTAAACNAATGTTTTCTACCGTGCAATATGCCAACTCAACCAAAC
>Resistance allele A/A
>Primer forward (underlined) (SEQ ID NO: 26)
AGCTTACAGGAAGGAGCGTIT
>Probe (double underlined)
Detect the resistant allele: TGGGTTGTCTCATATCG (SEQ ID NO: 27)
Detect the susceptible allele: AATGTGGGTTGTCTCGTA (SEQ ID NO: 28)
>Primer reverse (underlined)
GTTGGCATATTGCACGGTAGA (SEQ ID NO: 29)
Marker 7 at 149228421 bp on Spov3_chr3 (based on reference spinach_Viroflay_v3)
Target sequence (SEQ ID NO: 35)
TGGAGATTGTGTTTGACTCAGGTCTTGCACTTTGCTGGGTGTAGGATAGACACTT[T/A]CACACTTTCA
CACTTTCACTATCTGTCTTCTTATTGTTCAGTGTGCTTAAATGCTGTATTTGTATGT
>Resistance allele T/T
>Primer forward (underlined) (SEQ ID NO: 31)
GTTTGACTCAGGTCTTGCACTTT
>Probe (double underlined)
Detect the resistant allele: AGGATAGACACTTTCACA (SEQ ID NO: 32)
Detect the susceptible allele: TAGGATAGACACTTACACA (SEQ ID NO: 33)
>Primer reverse (underlined)
ACAGCATTTAAGCACACTGAACA (SEQ ID NO: 34)
Marker 8 at 149422498 bp on Spov3_chr3 (based on reference spinach_Viroflay_v3)
Target sequence (SEQ ID NO: 40)
TCCGTACTCNACTCACAAGGGATGTGCCAAAATCAAT[A/G]GTGTGCCTAAATCAGTTTCCCCCATTT
TCTTTCAAACCTCAATTACAGGCAAGTGTNGCCG
>Resistance allele A/A
>Primer forward (underlined) (SEQ ID NO: 36)
ACTCACAAGGGATGTGCCAA
>Probe (double underlined)
Detect the resistant allele: TGATTTAGGCACACTATTGAT (SEQ ID NO: 37)
Detect the susceptible allele: TTTAGGCACACCATTGATT (SEQ ID NO: 38)
>Primer reverse (underlined)
TGCCTGTAATTGAGGTTTGAAAGA (SEQ ID NO: 39)
Marker 9 at 149532142 bp on Spov3_chr3 (based on reference spinach_Viroflay_v3)
Target sequence (SEQ ID NO: 45)
GTTTTTTTCTGGGATGTAGAAGCTAAACAGGTTGGAAACAACCTCTCTGC[A/G]AATGCAGGGGNAAGGTTGCN
TACGTCCNACCCCCCTTACCCCNCTTCTTNCGGGAGCNTCTTTGAGGCAATGGGGTAATGATAATGATGAATGAT
AATGATGTAGAAGCTAAACAGGCTGTTCTTGAATATAGAGGAAAGTGACTGCACGGAGTTTATTNTTTG
>Resistance allele G/G
>Primer forward (underlined) (SEQ ID NO: 41)
GGGATGTAGAAGCTAAACAG
>Probe (double underlined)
Detect the resistant allele: AACCTCTCTGCGAATG (SEQ ID NO: 42)
Detect the susceptible allele: ACAACCTCTCTGCAA (SEQ ID NO: 43)
>Primer reverse (underlined)
ACTCCGTGCAGTCACTT (SEQ ID NO: 44)
Marker 10 at 149535556 bp on Spov3_chr3 (based on reference spinach_Viroflay_v3)
Target sequence (SEQ ID NO: 50)
GCAGTTTCGAGCTCTCAACAATGGCGTTTGGNCTTAGAATCTTCACACAAAGAACCAGCGACACC[A/G]TAGCACAA
CCAATTCTCGACGCCGACAACGGTGAAGTGCTTATGCACACTCANCCTTCCGTCTCNATTGTCCTCGGAAATCGCTCC
CCCGAATCAC
>Resistance allele A/A
>Primer forward (underlined) (SEQ ID NO: 46)
GCTCTCAACAATGGCGTTTGG
>Probe (double underlined)
Detect the resistant allele: AGCGACACCATAGCACA (SEQ ID NO: 47)
Detect the susceptible allele: AGCGACACCGTAGCAC (SEQ ID NO: 48)
>Primer reverse (underlined)
GGAGCGATTTCCGAGGACAA (SEQ ID NO: 49)
Marker 11 at 149579722 bp on Spov3_chr3 (based on reference spinach_Viroflay_v3)
Target sequence (SEQ ID NO: 55)
ATCAAGTNATTGCTGATCTCCCAGTAAGTGTTATGNTTACGTGCAATTATTCTGGTTAACCTTGAATATACTTNCATC
ATCCATCATAATCAAATAGGTTTATTATGAGGGTGTTATTTTTCAAAAATTAGGTTTCAACTGNNNNNANNCTGTGAT
TCCTGTCTAAGGAAAC[A/G]AGTCATTACATTGAATTTGGTGGTTGCGTGGAACCTATTTGTAA
>Resistance allele G/G
>Primer forward (underlined) (SEQ ID NO: 51)
TGCTGATCTCCCAGTAAGTGT
>Probe (double underlined)
Detect the resistant allele: TCAATGTAATGACTCGT (SEQ ID NO: 52)
Detect the susceptible allele: TTCAATGTAATGACTTGTT (SEQ ID NO: 53)
>Primer reverse (underlined)
GTTCCACGCAACCACCAA (SEQ ID NO: 54)

The information above is summarized in Table 5 below:

TABLE 5

| Marker No. | 1 | 2 | 3 | 4 | 5 | 6 | 7 | 8 | 9 | 10 | 11 |
|---|---|---|---|---|---|---|---|---|---|---|---|
| Resistance Allele¹ | A | A | A | G | C | A | T | A | G | A | G |
| Susceptible Allele | G | G | C | A | A | G | A | G | A | G | A |
| Target Sequence: | 5 | 10 | 15 | 20 | 25 | 30 | 35 | 40 | 45 | 50 | 55 |
| SEQ ID NO. | | | | | | | | | | | |
| SNP Position in | 77 | 71 | 50 | 73 | 79 | 55 | 56 | 38 | 51 | 66 | 173 |
| Target: nt | | | | | | | | | | | |
| Forward Primer: | 1 | 6 | 11 | 16 | 21 | 26 | 31 | 36 | 41 | 46 | 51 |
| SEQ ID NO. | | | | | | | | | | | |
| Reverse Primer: | 4 | 9 | 14 | 19 | 24 | 29 | 34 | 39 | 44 | 49 | 54 |
| SEQ ID NO. | | | | | | | | | | | |
| Probe (Resistant): | 2 | 7 | 12 | 17 | 22 | 27 | 32 | 37 | 42 | 47 | 52 |
| SEQ ID NO. | | | | | | | | | | | |
| Probe | 3 | 8 | 13 | 18 | 23 | 28 | 33 | 38 | 43 | 48 | 53 |
| (Susceptible): | | | | | | | | | | | |
| SEQ ID NO. | | | | | | | | | | | |

¹Resistance present in both heterozygotes and homozygotes

### Example 4. ADZ Peronospora Resistance

A second recombination designated “ADZ” was produced from the same wild Spinacia turkestanica source (accession KK016; CGN080741) following a similar approach as described above for ADQ (Example 2), but using a different cultivated S. oleracea line as the recurrent parent. However, both ADQ and ADZ recurrent parents have the same fixed Peronospora effusa resistance.

ADZ has the same resistance spectrum as ADQ against all tested isolates (e.g., dominant, qualitative resistance to Pe: 1-18 in homozygous and heterozygous plants) without linkage drag or other adverse phenotypes (e.g., dwarfism), and further has the same haplotype as ADQ (as described above in Example 3). Marker 11 is essentially diagnostic for both ADQ and ADZ resistances, and optionally one or more of markers 1 to 10 can also be used to identify spinach plants comprising the ADZ Peronospora resistance, to follow the resistance for introgression into new lines, and the like.

S. oleracea line 21BNL002487 containing the ADZ recombination was deposited with NCIMB on 21 Oct. 2022 under Deposit No. NCIMB 44060. This line is fixed (i.e., homozygous) for the P. effusa resistance, but is otherwise still segregating at many loci.

Although the present invention has been described with reference to specific details of certain embodiments thereof, it is not intended that such details should be regarded as limitations upon the scope of the claimed invention.

1. A Spinacia oleracea plant, optionally a cultivated S. oleracea plant, with enhanced resistance to Peronospora effusa comprising an introgressed sequence that confers a qualitative and dominant resistance to Peronospora effusa, wherein said introgressed sequence is located on chromosome 3 and comprises a G genotype in the heterozygous or homozygous state for SNP marker 11 in SEQ ID NO: 55;
wherein the resistance of the plant to Peronospora effusa is enhanced as compared with a S. oleracea plant lacking said introgressed sequence.

2. The plant according to claim 1, wherein said introgressed sequence further comprises a Peronospora resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45; and/or
j) an A genotype in the heterozygous or homozygous state for SNP marker 10 in SEQ ID NO: 50.

3. The plant according to claim 2, wherein the introgressed sequence comprises the Peronospora resistance-associated genotype at two or more, three or more, four or more, five or more, six or more, seven or more, eight or more, nine or more, or all of the SNP markers of a) to j).

4. The plant according to claim 2, wherein the introgressed sequence comprises the Peronospora resistance-associated genotype at two or more, three or more, four or more, five or more, six or more, seven or more, or all of the SNP markers of c) to j).

5. The plant according to claim 2, wherein the introgressed sequence comprises the Peronospora resistance-associated genotype at any of the following combinations of SNP markers:
i. 11 and 1;
ii. 11 and 2;
iii. 11 and 3;
iv. 11 and 4;
v. 11 and 5;
vi. 11 and 6;
vii. 11 and 7;
viii. 11 and 8;
ix. 11, 10, 9 and 8;
x. 11, 10, 9, 8 and 7;
xi. 11, 10, 9, 8, 7 and 6;
xii. 11, 10, 9, 8, 7, 6 and 5;
xiii. 11, 10, 9, 8, 7, 6, 5 and 4;
xiv 11, 10, 9, 8, 7, 6, 5, 4 and 3;
xv. 11, 10, 9, 8, 7, 6, 5, 4, 3 and 2;
xvi. 11, 10, 9, 8, 7, 6, 5, 4, 3 and 1; or
xvii. 11, 10, 9, 8, 7, 6, 5, 4, 3, 2 and 1.

6. The plant according to claim 1, wherein said introgressed sequence confers resistance against at least Peronospora effusa races Pe: 1-18.

7. The plant according to claim 1, wherein the plant is heterozygous for the introgressed sequence.

8. The plant according to claim 1, wherein the plant is homozygous for the introgressed sequence.

9. The plant according to claim 1, wherein said introgressed sequence comprises one or more of SEQ ID NO: 5, SEQ ID NO: 10, SEQ ID NO: 15, SEQ ID NO: 20, SEQ ID NO: 25, SEQ ID NO: 30, SEQ ID NO: 35, SEQ ID NO: 40, SEQ ID NO: 45, SEQ ID NO: 50, and/or SEQ ID NO: 55, or a sequence that is at least 80% identical to one or more of the foregoing sequences and comprises the indicated SNP marker genotype.

10. The plant according to claim 1, wherein:
the A genotype for SNP marker 1 can be identified in a PCR by amplification of a nucleic acid fragment with a pair of oligonucleotide primers: forward primer of SEQ ID NO: 1 and reverse primer of SEQ ID NO: 4, and probe of SEQ ID NO: 2;
the A genotype for SNP marker 2 can be identified in a PCR by amplification of a nucleic acid fragment with a pair of oligonucleotide primers: forward primer of SEQ ID NO: 6 and reverse primer of SEQ ID NO: 9, and probe of SEQ ID NO: 7;
the A genotype for SNP marker 3 can be identified in a PCR by amplification of a nucleic acid fragment with a pair of oligonucleotide primers: forward primer of SEQ ID NO: 11 and reverse primer of SEQ ID NO: 14, and probe of SEQ ID NO: 12;
the G genotype for SNP marker 4 can be identified in a PCR by amplification of a nucleic acid fragment with a pair of oligonucleotide primers: forward primer of SEQ ID NO: 16 and reverse primer of SEQ ID NO: 19, and probe of SEQ ID NO: 17;
the C genotype for SNP marker 5 can be identified in a PCR by amplification of a nucleic acid fragment with a pair of oligonucleotide primers: forward primer of SEQ ID NO: 21 and reverse primer of SEQ ID NO: 24, and probe of SEQ ID NO: 22;
the A genotype for SNP marker 6 can be identified in a PCR by amplification of a nucleic acid fragment with a pair of oligonucleotide primers: forward primer of SEQ ID NO: 26 and reverse primer of SEQ ID NO: 29, and probe of SEQ ID NO: 27;
the T genotype for SNP marker 7 can be identified in a PCR by amplification of a nucleic acid fragment with a pair of oligonucleotide primers: forward primer of SEQ ID NO: 31 and reverse primer of SEQ ID NO: 34, and probe of SEQ ID NO: 32;
the A genotype for SNP marker 8 can be identified in a PCR by amplification of a nucleic acid fragment with a pair of oligonucleotide primers: forward primer of SEQ ID NO: 36 and reverse primer of SEQ ID NO: 39, and probe of SEQ ID NO: 37;
the G genotype for SNP marker 9 can be identified in a PCR by amplification of a nucleic acid fragment with a pair of oligonucleotide primers: forward primer of SEQ ID NO: 41 and reverse primer of SEQ ID NO: 44, and probe of SEQ ID NO: 42;
the A genotype for SNP marker 10 can be identified in a PCR by amplification of a nucleic acid fragment with a pair of oligonucleotide primers: forward primer of SEQ ID NO: 46 and reverse primer of SEQ ID NO: 49, and probe of SEQ ID NO: 47; and
the G genotype for SNP marker 11 can be identified in a PCR by amplification of a nucleic acid fragment with a pair of oligonucleotide primers: forward primer of SEQ ID NO: 51 and reverse primer of SEQ ID NO: 54, and probe of SEQ ID NO: 52.

11. The plant of claim 1, wherein said introgressed sequence is comprised in S. oleracea line 21BNL002487 or S. oleracea line 21BNL002472, representative seed of S. oleracea line 21BNL002487 and S. oleracea line 21BNL002472 having been deposited with the NCIMB under Accession No. NCIMB 43893 and Accession No. NCIMB 44060, respectively.

12. The plant of claim 1, wherein the plant is an inbred, a dihaploid or a hybrid plant.

13. A plant of Spinacia oleracea line 21BNL002487 or S. oleracea line 21 BNL002472, representative seed of S. oleracea line 21BNL002487 and S. oleracea line 21BNL002472 having been deposited with the NCIMB under Accession No. NCIMB 43893 and Accession No. NCIMB 44060, respectively.

14. An F1 progeny plant of the plant of claim 13.

15. A seed that produces a plant according to claim 1.

16. A plant part of the plant according to claim 1.

17. A method of producing spinach seed, the method comprising growing a spinach plant from the seed of claim 15, and allowing the plant to produce further spinach seed.

18. A method for producing a spinach plant with enhanced resistance to Peronospora effusa, the method comprising:
crossing a first S. oleracea plant according to claim 1 with a second spinach plant lacking said introgressed sequence conferring resistance to Peronospora effusa to produce progeny plants; and
selecting a progeny plant comprising said introgressed sequence conferring resistance to Peronospora effusa, said selecting step comprising detecting in the plant the presence of a resistance-associated genotype at one or more SNP markers in the chromosome interval defined by SNP marker 11 to 1, optionally SNP marker 11 to 3.

19. The method according to claim 18, wherein said selecting step comprises detecting the presence of a resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45;
j) an A genotype in the heterozygous or homozygous state for SNP marker 10 in SEQ ID NO: 50; and/or
k) a G genotype in the heterozygous or homozygous state for SNP marker 11 in SEQ ID NO: 55,
thereby producing a plant with enhanced resistance to Peronospora effusa.

20. The method according to claim 19, wherein the method comprises detecting the presence of the Peronospora resistance-associated genotype at two or more, three or more, four or more, five or more, six or more, seven or more, eight or more, nine or more, ten or more, or all of the SNP markers of a) to k).

21. The method according to claim 19, wherein the method comprises detecting the presence of the Peronospora resistance-associated genotype at two or more, three or more, four or more, five or more, six or more, seven or more, eight or more, or all of the SNP markers of c) to k).

22. The method according to claim 18, wherein the method further comprises:
selfing the selected progeny or crossing the selected progeny plant with another spinach plant to produce further progeny.

23. The method according to claim 22, wherein a further progeny plant is selected and selfed and/or crossed for 2 to 10 more generations.

24. A method for producing a F1 hybrid spinach plant with enhanced resistance to Peronospora effusa, the method comprising crossing an inbred S. oleracea plant, which is a plant according to claim 1, with a different inbred S. oleracea plant to produce a F1 hybrid progeny plant.

25. The method according to claim 24, wherein the different inbred S. oleracea plant does not comprise the introgressed sequence conferring enhanced resistance to Peronospora effusa, optionally wherein the different inbred S. oleracea plant is susceptible to Peronospora effusa.

26. The method according to claim 24, wherein said method further comprises detecting in the F1 hybrid progeny plant the presence of a resistance-associated genotype at one or more SNP markers in the chromosome interval defined by SNP marker 11 to 1, optionally SNP marker 11 to 3.

27. The method according to claim 24, wherein said method further comprises detecting in the F1 hybrid progeny plant the presence of a resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45;
j) an A genotype in the heterozygous or homozygous state for SNP marker 10 in SEQ ID NO: 50; and/or
k) a G genotype in the heterozygous or homozygous state for SNP marker 11 in SEQ ID NO: 55.

28. The method according to claim 27, wherein the method comprises detecting the presence of the Peronospora resistance-associated genotype at two or more, three or more, four or more, five or more, six or more, seven or more, eight or more, nine or more, ten or more, or all of the SNP markers of a) to k), optionally at two or more, three or more, four or more, five or more, six or more, seven or more, eight or more, or all of the SNP markers of c) to k).

29. A method for identifying a spinach plant with enhanced resistance to Peronospora effusa, said method comprising the step of detecting in the plant the presence of a resistance-associated genotype at one or more SNP markers in the chromosome interval defined by SNP marker 11 to 1, optionally SNP marker 11 to 3.

30. The method according to claim 29, wherein said method comprises detecting the presence of a resistance-associated genotype at one or more of the following SNP markers:
a) an A genotype in the heterozygous or homozygous state for SNP marker 1 in SEQ ID NO: 5;
b) an A genotype in the heterozygous or homozygous state for SNP marker 2 in SEQ ID NO: 10;
c) an A genotype in the heterozygous or homozygous state for SNP marker 3 in SEQ ID NO: 15;
d) a G genotype in the heterozygous or homozygous state for SNP marker 4 in SEQ ID NO: 20;
e) a C genotype in the heterozygous or homozygous state for SNP marker 5 in SEQ ID NO: 25;
f) an A genotype in the heterozygous or homozygous state for SNP marker 6 in SEQ ID NO: 30;
g) a T genotype in the heterozygous or homozygous state for SNP marker 7 in SEQ ID NO: 35;
h) an A genotype in the heterozygous or homozygous state for SNP marker 8 in SEQ ID NO: 40;
i) a G genotype in the heterozygous or homozygous state for SNP marker 9 in SEQ ID NO: 45;
j) an A genotype in the heterozygous or homozygous state for SNP marker 10 in SEQ ID NO: 50; and/or
k) a G genotype in the heterozygous or homozygous state for SNP marker 11 in SEQ ID NO: 55,
thereby identifying a spinach plant with enhanced resistance to Peronospora effusa.

31. The method according to claim 30, wherein the method comprises detecting the presence of the Peronospora resistance-associated genotype at two or more, three or more, four or more, five or more, six or more, seven or more, eight or more, nine or more, ten or more, or all of the SNP markers of a) to k), optionally at two or more, three or more, four or more, five or more, six or more, seven or more, eight or more, or all of the SNP markers of c) to k).

32. The method according to claim 29, wherein said method further comprises selecting a spinach plant comprising said resistance-associated genotype at one or more SNP markers, and crossing the selected spinach plant with a second spinach plant to produce a progeny spinach plant that comprises said resistance-associated genotype at the one or more SNP markers and has enhanced resistance to Peronospora effusa.

33. The method according to claim 18, wherein the method comprises detecting the presence of the Peronospora resistance-associated genotype at any of the following combinations of SNP markers:
i. 11 and 1;
ii. 11 and 2;
iii. 11 and 3;
iv. 11 and 4;
v. 11 and 5;
vi. 11 and 6;
vii. 11 and 7;
viii. 11 and 8;
ix. 11, 10, 9 and 8;
x. 11, 10, 9, 8 and 7;
xi. 11, 10, 9, 8, 7 and 6;
xii. 11, 10, 9, 8, 7, 6 and 5;
xiii. 11, 10, 9, 8, 7, 6, 5 and 4;
xiv 11, 10, 9, 8, 7, 6, 5, 4 and 3;
xv. 11, 10, 9, 8, 7, 6, 5, 4, 3 and 2;
xvi. 11, 10, 9, 8, 7, 6, 5, 4, 3 and 1; or
xvii. 11, 10, 9, 8, 7, 6, 5, 4, 3, 2 and 1.

34. The method according to claim 18, wherein the spinach plant that is produced or identified has resistance against at least Peronospora effusa races Pe: 1-18.

35. The method according to claim 18, wherein the plant is heterozygous for the introgressed sequence.

36. The method according to claim 18, wherein the plant is homozygous for the introgressed sequence.

37. The method according to claim 18, wherein said method comprises detecting the presence or absence of one or more of SEQ ID NO: 5, SEQ ID NO: 10, SEQ ID NO: 15, SEQ ID NO: 20, SEQ ID NO: 25, SEQ ID NO: 30, SEQ ID NO: 35, SEQ ID NO: 40, SEQ ID NO: 45, SEQ ID NO: 50, and/or SEQ ID NO: 55, or a sequence that is at least 80% identical to one or more of the foregoing sequences and comprises the Peronospora resistance-associated SNP marker genotype.

38. The method according to claim 18, wherein:
the A genotype for SNP marker 1 can be identified in a PCR by amplification of a nucleic acid fragment with a pair of oligonucleotide primers: forward primer of SEQ ID NO: 1 and reverse primer of SEQ ID NO: 4, and probe of SEQ ID NO: 2;
the A genotype for SNP marker 2 can be identified in a PCR by amplification of a nucleic acid fragment with a pair of oligonucleotide primers: forward primer of SEQ ID NO: 6 and reverse primer of SEQ ID NO: 9, and probe of SEQ ID NO: 7;
the A genotype for SNP marker 3 can be identified in a PCR by amplification of a nucleic acid fragment with a pair of oligonucleotide primers: forward primer of SEQ ID NO: 11 and reverse primer of SEQ ID NO: 14, and probe of SEQ ID NO: 12;
the G genotype for SNP marker 4 can be identified in a PCR by amplification of a nucleic acid fragment with a pair of oligonucleotide primers: forward primer of SEQ ID NO: 16 and reverse primer of SEQ ID NO: 19, and probe of SEQ ID NO: 17;
the C genotype for SNP marker 5 can be identified in a PCR by amplification of a nucleic acid fragment with a pair of oligonucleotide primers: forward primer of SEQ ID NO: 21 and reverse primer of SEQ ID NO: 24, and probe of SEQ ID NO: 22;
the A genotype for SNP marker 6 can be identified in a PCR by amplification of a nucleic acid fragment with a pair of oligonucleotide primers: forward primer of SEQ ID NO: 26 and reverse primer of SEQ ID NO: 29, and probe of SEQ ID NO: 27;
the T genotype for SNP marker 7 can be identified in a PCR by amplification of a nucleic acid fragment with a pair of oligonucleotide primers: forward primer of SEQ ID NO: 31 and reverse primer of SEQ ID NO: 34, and probe of SEQ ID NO: 32;
the A genotype for SNP marker 8 can be identified in a PCR by amplification of a nucleic acid fragment with a pair of oligonucleotide primers: forward primer of SEQ ID NO: 36 and reverse primer of SEQ ID NO: 39, and probe of SEQ ID NO: 37;
the G genotype for SNP marker 9 can be identified in a PCR by amplification of a nucleic acid fragment with a pair of oligonucleotide primers: forward primer of SEQ ID NO: 41 and reverse primer of SEQ ID NO: 44, and probe of SEQ ID NO: 42;
the A genotype for SNP marker 10 can be identified in a PCR by amplification of a nucleic acid fragment with a pair of oligonucleotide primers: forward primer of SEQ ID NO: 46 and reverse primer of SEQ ID NO: 49, and probe of SEQ ID NO: 47; and
the G genotype for SNP marker 11 can be identified in a PCR by amplification of a nucleic acid fragment with a pair of oligonucleotide primers: forward primer of SEQ ID NO: 51 and reverse primer of SEQ ID NO: 54, and probe of SEQ ID NO: 52.
